(19) United States
(12) Patent Application Publication
(10) Pub. No.: US 2013/0030487 A1
(43) Pub. Date: Jan. 31, 2013

(54) DEVICES, SYSTEMS AND METHODS TO INCREASE COMPLIANCE WITH A PREDETERMINED VENTRICULAR ELECTRICAL ACTIVATION PATTERN

(71) Applicants: Allen J. Keel, San Francisco, CA (US); Kyungmoo Ryu, Palmdale, CA (US); Stuart Rosenberg, Castaic, CA (US)

(72) Inventors: Allen J. Keel, San Francisco, CA (US); Kyungmoo Ryu, Palmdale, CA (US); Stuart Rosenberg, Castaic, CA (US)

(21) Appl. No.: 13/194,716

(22) Filed: Jul. 29, 2011

(51) Int. Cl. A61N 1/368

(52) U.S. Cl. 607/18

(57) ABSTRACT

Described herein are implantable systems and devices, and methods for use therewith, that can be used to increase compliance with a predetermined preferred ventricular electrical activation pattern. Such implantable systems preferably includes a first lead having at least one electrode implantable in a right ventricular (RV) chamber, and a second lead having at least two electrodes implantable in a left ventricular (LV) chamber. A plurality of different sensing vectors are used to obtain a plurality of IEGMs that collectively enable electrical activations to be detected in at least the RV chamber and at at least two separate regions of the LV chamber. The IEGMs can be obtained while the patient's LV chamber is not being paced, or during bi-ventricular (BiV) pacing that includes pacing at only a single site within the LV chamber. An actual ventricular electrical activation pattern is determined based on the plurality of IEGMs. Additionally, there is a determination of whether the actual ventricular electrical activation pattern matches the predetermined preferred ventricular electrical activation pattern. If the actual ventricular electrical activation pattern does not match the predetermined preferred ventricular electrical activation pattern, then multisite LV (MSLV) pacing is delivered to achieve the predetermined preferred ventricular electrical activation pattern.

### RELATED APPLICATIONS

The present application is related to the following commonly invented and commonly assigned patent applications, each of which is filed the same day as the present application, and each of which is incorporated herein by reference: U.S. patent application Ser. No. ______, entitled DEVICES, SYSTEMS AND METHODS TO MONITOR AND TREAT HEART FAILURE (HF) (Attorney Docket No. A11P3006); U.S. patent application Ser. No. ______, entitled DEVICES, SYSTEMS AND METHODS TO PERFORM ARRHYTHMIA DISCRIMINATION BASED ON R-R INTERVAL STABILITY CORRESPONDING TO A PLURALITY OF VENTRICULAR REGIONS (Attorney Docket No. A11P3008); and U.S. patent application Ser. No. ______, entitled DEVICES, SYSTEMS AND METHODS TO PERFORM ARRHYTHMIA DISCRIMINATION BASED ON THE ATRIAL AND VENTRICULAR ACTIVATION TIMES (Attorney Docket No. A11P3009).

### FIELD OF THE INVENTION

Embodiments of the present invention generally relate to implantable systems and devices, and methods for use therewith, that can be used to monitor and treat heart failure, increase compliance with a predetermined preferred ventricular electrical activation pattern, and/or perform arrhythmia discrimination.

### BACKGROUND

Heart failure (HF) is a debilitating, end-stage disease in which abnormal function of the heart leads to inadequate blood flow to fulfill the needs of the body's tissues. Typically, the heart loses propulsive power because the cardiac muscle loses capacity to stretch and contract. Often, the ventricles do not adequately fill with blood between heartbeats and the valves regulating blood flow may become leaky, allowing regurgitation or backflow of blood. The impairment of arterial circulation deprives vital organs of oxygen and nutrients. Fatigue, weakness, and inability to carry out daily tasks may result.

Not all HF patients suffer debilitating symptoms immediately. Some may live actively for years. Yet, with few exceptions, the disease is relentlessly progressive. As HF progresses, it tends to become increasingly difficult to manage. Even the compensatory responses it triggers in the body may themselves eventually complicate the clinical prognosis. For example, when the heart attempts to compensate for reduced cardiac output, it adds muscle causing the ventricles to grow in volume in an attempt to pump more blood with each heartbeat. This places a still higher demand on the heart's oxygen supply. If the oxygen supply falls short of the growing demand, as it often does, further injury to the heart may result. The additional muscle mass may also stiffen the heart walls to hamper rather than assist in providing cardiac output.

Current standard treatment for HF is typically centered around medical treatment using ACE inhibitors, diuretics, and digitalis. It has also been demonstrated that aerobic exercise may improve exercise tolerance, improve quality of life, and decrease symptoms. Cardiac surgery has also been performed on a small percentage of patients with particular etiologies. Although advances in pharmacological therapy have significantly improved the survival rate and quality of life of patients, some HF patients are refractory to drug therapy, have a poor prognosis and limited exercise tolerance. In recent years cardiac pacing, in particular Cardiac Resynchronization Therapy (CRT), has emerged as an effective treatment for many patients with drug-refractory HF.

HF patients require close medical management to reduce morbidity and mortality. Because the disease status evolves with time, frequent physician follow-up examinations are often necessary. At follow-up, the physician may make adjustments to the drug regimen in order to optimize therapy. This conventional approach of periodic follow-up may be less satisfactory for HF, in which acute, life-threatening exacerbations can develop between physician follow-up examinations. It is well known among clinicians that if a developing exacerbation is recognized early, it can be more easily and inexpensively terminated, typically with a modest increase in oral diuretic. However, if it develops beyond the initial phase, an acute HF exacerbation becomes difficult to control and terminate. Hospitalization in an intensive care unit is often required. It is during an acute exacerbation of HF that many patients succumb to the disease. Early identification may also allow for pacing therapy from an implanted pulse generator.

In view of the above, it would be beneficial if a patient's HF condition can be chronically monitored. Additionally, it would be beneficial if therapy can be automatically delivered and adjusted to improve HF treatment. Further, it would be beneficial to facilitate the early termination of a developing HF exacerbation.

A properly time ordered ventricular electrical activation sequence and proper inter-ventricular and intra-ventricular delays (which can collectively be referred to as a proper ventricular electrical activation pattern) enable efficient contractions of the ventricular chambers of the heart, thereby contributing to efficient blood perfusion through both the lungs and the systemic circulation. However, in patients with right bundle branch block (RBBB), left bundle branch block (LBBB), or other ventricular conduction abnormalities, their ventricular electrical activation sequence and inter-ventricular and intra-ventricular delays are often adversely affected, which may adversely affect blood perfusion and, more generally, may adversely affect cardiac mechanical function. Accordingly, it would be desirable to be able to detect when a patient's ventricular electrical activation pattern deviates from a predetermined preferred pattern, and to increase compliance with the predetermined preferred pattern.

In a normal heart, cells of the sinoatrial node (SA node) spontaneously depolarize and thereby initiate an action potential. This action potential propagates rapidly through the atria (which contract), slowly through the atrioventricular node (AV node), the atrioventricular bundle (AV bundle or His bundle) and then to the ventricles, which causes ventricular contraction. This sequence of events is known as normal sinus rhythm (NSR). Thus, in a normal heart, ventricular rhythm relies on conduction of action potentials through the AV node and AV bundle.

Rhythms that do not follow the sequence of events described above are known as arrhythmias. Those that result in a heart rate slower than normal are known as bradyarrhythmias; those that result in a faster heart rate than normal are called tachyarrhythmias. Tachyarrhythmias are further classified as supraventricular tachyarrhythmias (SVTs) and ventricular tachyarrhythmia (VT). SVTs are generally characterized by abnormal rhythms that may arise in the atria or the atrioventricular node (AV node). Additionally, there are various types of different SVTs and various types of VTs that can be characterized. For example, a paroxysmal SVT can exhibit heart rates between approximately 140 beats per minute (bpm) and approximately 250 bpm. However, the most common SVTs are typically atrial flutter (AFL) and atrial fibrillation (AF). In addition, many SVTs involve the AV node, for example, AV nodal reentry tachycardia (AVNRT) where an electrical loop or circuit includes the AV node. Another type of SVT is an AV reentrant tachycardia (AVRT), where an AV reentrant circuit typically involves the AV node and an aberrant conducting bundle known as an accessory pathway that connects a ventricle to an atrium.

Atrial flutter (AFL) can result when an early beat triggers a “circus circular current” that travels in regular cycles around the atrium, pushing the atrial rate up to approximately 250 bpm to approximately 350 bpm. The atrioventricular node between the atria and ventricles will often block one of every two beats, keeping the ventricular rate at about 125 bpm to about 175 bpm. This is the pulse rate that will be felt, even though the atria are beating more rapidly. At this pace, the ventricles will usually continue to pump blood relatively effectively for many hours or even days. A patient with underlying heart disease, however, may experience chest pain, faintness, or even HF as a result of the continuing increased stress on the heart muscle. In some individuals, the ventricular rate may also be slower if there is increased block of impulses in the AV node, or faster if there is little or no block.

If the cardiac impulse fails to follow a regular circuit and divides along multiple pathways, a chaos of uncoordinated beats results, producing AF. AF commonly occurs when the atrium is enlarged (usually because of heart disease). In addition, it can occur in the absence of any apparent heart disease. In AF, the atrial rate can increase to more than 350 bpm and cause the atria to fail to pump blood effectively. Under such circumstances, the ventricular beat may also become haphazard, producing a rapid irregular pulse. Although AF may cause the heart to lose approximately 20 to 30 percent of its pumping effectiveness, the volume of blood pumped by the ventricles usually remains within the margin of safety, again because the atrioventricular node blocks out many of the chaotic beats. Hence, during AF, the ventricles may contract at a lesser rate than the atria, for example, of approximately 125 bpm to approximately 175 bpm.

Overall, SVTs are not typically immediately life threatening when compared to ventricular arrhythmias, examples of which are discussed below.

Ventricular arrhythmias, which originate in the ventricles, include ventricular tachycardia (VT) and ventricular fibrillation (VF). Ventricular arrhythmias are often associated with rapid and/or chaotic ventricular rhythms. For example, sustained VT can lead to VF. In sustained VT, consecutive impulses arise from the ventricles at a rate of 100 bpm or more. Such activity may degenerate further into disorganized electrical activity known as ventricular fibrillation (VF). In VF, disorganized action potentials can cause the myocardium to quiver rather than contract. Such chaotic quivering can greatly reduce the heart's pumping ability. Indeed, approximately two-thirds of all deaths from arrhythmia are caused by VF. A variety of conditions such as, but not limited to, hypoxia, ischemia, pharmacologic therapy (e.g., sympathomimetics), and asynchronous pacing may promote onset of ventricular arrhythmia. Further, there are various different types of VT, including, e.g., monomorphic VT and polymorphic VT, for which different types of therapy may be appropriate.

It has been common practice for an implantable cardioverter defibrillator (ICD) to monitor heart rate, or more commonly the ventricular rate, of a patient and classify the cardiac condition of the patient based on this heart rate. For example, a tachyarrhythmia may be defined as any rate in a range above a designated threshold. This range is then divided into ventricular tachycardia and ventricular fibrillation zones. The ventricular tachycardia zone may be further divided into slow ventricular tachycardia and fast ventricular tachycardia zones.

As described above, both SVTs and ventricular arrhythmias may lead to ventricular rates in excess of 100 bpm. In other words, ventricular rates of SVTs can overlap with rates of tachycardias of ventricular origin. These SVTs are often well tolerated and require no intervention. Further, physically active patients can have heart rates during exercise that overlap with their tachycardia rates. Accordingly, discrimination of VT from SVT, including increased heart rates due to exercise, may require more than just knowledge of a patient's ventricular rate. In other words, using heart rate as the sole criterion to classify the cardiac condition of a patient is often not sufficient.

To improve the specificity and accuracy of arrhythmia characterization, many ICDs can also examine the morphology of an intracardiac electrogram (IEGM), in addition to the heart rate. The shape of an intracardiac complex can include information on the origin and sequence of the heart's electrical activity. A normal intracardiac complex traverses the AV node and is conducted by specialized cardiac tissue throughout the ventricles. This results in a distinctive complex morphology. A tachycardia of ventricular origin often has a different morphology due to its ectopic origin and conductance through cardiac muscle tissue. As such, in addition to monitoring heart rate, some ICDs are capable of performing morphology discrimination to classify the cardiac condition of the patient. For example, a template based on the morphology of a “known” signal can be stored in the ICD. The “known” signal can be, for example, a signal collected during a period where a patient is known to exhibit a normal sinus rhythm. By comparing the morphology characteristics (e.g., number, amplitude, sequence and/or polarity of waveform peaks, as well as the area of the peaks) of an arrhythmia to the template, the ICD can calculate the match (or lack thereof) between the waveforms. For a further description of morphology discrimination, refer to U.S. Pat. No. 5,240,009 (Williams), entitled “Medical Device with Morphology Discrimination” and to U.S. Pat. No. 5,779,645 (Olson et al.) entitled “System and Method for Waveform Morphology Comparison,” which patents are hereby incorporated by reference. These are just a few examples of morphology discriminator algorithms and parameters, which are not intended to be limiting.

Sudden onset and interval stability (also know as rate stability), are examples of other factors that can be monitored to improve the specificity of arrhythmia characterization. Also, the relationship between ventricular rate (V) and atrial rate (A) can be used to characterize an arrhythmia. For example, this can be part of a rate branch algorithm, which, depending on V and A, may follow one of three branches: a V<A rate branch; a V=A (within a specified tolerance) rate branch; and a V>A rate branch. If V<A, then morphology discrimination and/or interval stability may be available to distinguish VT from AF or AFL. If A and V are essentially the same (within a certain tolerance), then morphology discrimination and/or sudden onset may be available to distinguish VT from sinus tachycardia. If V>A, then an arrhythmia may be characterized as VT. Also, specific branches can be turned on or off. For example, if V is greater than the tachycardia threshold but essentially the same as A, and the V=A branch is turned off, then the algorithm can cause the V>A branch to be followed, and the arrhythmia may be classified as VT. Additional details of an exemplary rate branch algorithm are provided in U.S. Pat. No. 6,636,764 (Fain et al.), entitled “Safety Backup in Arrhythmia Discrimination Algorithm,” which is incorporated herein by reference. Also, atrioventricular association (AVA) can also be used to distinguish AFL from VT. In an exemplary AVA algorithm, the AV interval is measured from each ventricular sensed event to its preceding atrial event and an AVA Delta is then calculated as the difference between the second longest AV interval and the second shortest AV interval in a recent group of intervals. If the measured AVA Delta is less than a programmable AVA threshold parameter, the AV intervals are considered stable, which is indicative of SVT. If the measured AVA Delta is greater than or equal to a programmable AVA threshold parameter, the AV intervals are considered unstable, which is indicative of VT. More generally, the relative rate of the atria and ventricles and/or the timing relationship between atrial and ventricular events can be considered.

Typically an ICD is programmed to provide a therapy in response to an arrhythmia being detected, where the type of therapy corresponds to the type of arrhythmia that the ICD believes it has detected. For example, VT may be treated with a therapy consisting of low-energy pacing pulses designed to capture the ventricles. This therapy is referred to as ventricular anti-tachycardia pacing therapy (V-ATP). VT may also be treated with relatively low energy, synchronized cardioversion shocks. VF, on the other hand, is typically treated more aggressively with high energy shocks. SVT may not be treated, or may be treated using atrial ATP (A-ATP) or atrial defibrillation. Quite often, SVT is treated using medication, or ablation.

Inappropriate therapy is a huge problem for ICD patients. Inappropriate therapies, specifically inappropriate shocks cause great suffering among the ICD patient population. Patients receive a potentially life-saving device (the ICD) only to find out that it sometimes malfunctions and inflicts both pain and harm without any warning whatsoever. For a secondary prevention patient, this might be bearable since he or she has experienced and been saved from a lethal tachyarrhythmia in the past. For the primary prevention patient, however, without a history of arrhythmias who receives the ICD only based on a risk score, this is more difficult to bear.

Today only a few of the patients who are candidates for an ICD receive one. This is mostly an economic issue; although several studies indicate that an ICD is a cost effective treatment in indicated patients. However, if the performance of the devices improves, the willingness to put in an ICD, especially in young primary prevention patients, will increase.

Despite the numerous arrhythmia discrimination techniques that exist, examples of which were provided above, delivery of inappropriate therapy remains a major problem with ICDs today. For example, a common cause of inappropriate shocks in ICD devices are atrial arrhythmias that are conducted to the ventricles at a high rate. Accordingly, there is still a need for new, and preferably improved, arrhythmia discrimination techniques.

### SUMMARY

Certain embodiments of the present invention generally relate to implantable systems and devices, and methods for use therewith, that can be used to monitor and treat HF. Such implantable systems preferably includes a lead having at least two electrodes implantable in a patient's left ventricular (LV) chamber. A plurality of different sensing vectors are used to obtain a plurality of IEGMs each of which is indicative of an evoked response at a corresponding different region of the LV chamber. For each of the IEGMs, there is a determination of one or more evoked response metrics indicative of a localized cardiac function at the corresponding region of the LV chamber. The evoked response metrics can be, e.g., paced depolarization integral (PDI) and/or maximum upward slope of an R-wave, but are not limited thereto. The patient's HF condition is monitored based on the localized cardiac function at the plurality of different regions of the LV chamber as determined based on the one or more evoked response metrics determined for each of the IEGMs. In specific embodiments, the above described steps can be repeated from time to time to thereby monitor changes in the patient's HF condition based on changes in the localized cardiac function at the plurality of different regions of the LV chamber as determined based on the one or more evoked response metrics determined for each of the IEGMs.

In specific embodiments, one or more LV pacing sites are selected for delivering cardiac resynchronization therapy (CRT) pacing in dependence on the localized cardiac function at the plurality of different regions of the patient's LV chamber, and more generally, based on the patient's HF condition. This can include selecting one or more LV pacing sites that improve the patient's HF condition. This can also include selecting one or more LV pacing sites that increase a similarity between evoked response metrics indicative of the localized cardiac function at a first region of the LV chamber and corresponding evoked response metrics indicative of the localized cardiac function at a second region of the LV chamber. In a specific embodiment, the selected LV pacing site for delivering CRT pacing is the region of the LV chamber that is identified, based on evoked response metrics, as having the lowest cardiac function. Alternatively, the selected LV pacing site for delivering CRT pacing is the region of the LV chamber that is identified, based on evoked response metrics, as having the highest cardiac function. Additionally, or alternatively, an alert can be selectively triggered based on the patient's HF condition, and/or information indicative of the patient's HF condition can be saved.

Other embodiments of the present invention generally relate to implantable systems and devices, and methods for use therewith, that can be used for increasing compliance with a predetermined preferred ventricular electrical activation pattern. Such implantable systems preferably includes a first lead having at least one electrode implantable in a right ventricular (RV) chamber, and a second lead having at least two electrodes implantable in an LV chamber. A plurality of different sensing vectors are used to obtain a plurality of IEGMs that collectively enable electrical activations to be detected in at least the RV chamber and at at least two separate regions of the LV chamber. The IEGMs can be obtained while the patient's LV chamber is not being paced, or during bi-ventricular (BiV) pacing that includes pacing at only a single site within the LV chamber. An actual ventricular electrical activation pattern is determined based on the plurality of IEGMs. Additionally, there is a determination of whether the actual ventricular electrical activation pattern matches the predetermined preferred ventricular electrical activation pattern. If the actual ventricular electrical activation pattern does not match the predetermined preferred ventricular electrical activation pattern, then multisite LV (MSLV) pacing is delivered to achieve the predetermined preferred ventricular electrical activation pattern.

The predetermined preferred ventricular electrical activation pattern can include a predetermined preferred ventricular electrical activation sequence (e.g., an LV1-LV2-RV sequence, or an RV-LV1-LV2 sequence). Additionally, the predetermined preferred ventricular electrical activation pattern can include at least two predetermined preferred ventricular electrical activation delays, which preferably includes at least one predetermined preferred intra-ventricular delay (e.g., an LV1-LV2 delay). The predetermined preferred ventricular electrical activation delays can also include a predetermined preferred inter-ventricular delay (e.g., an RV-LV1 delay, or an LV2-RV delay).

Still other embodiments of the present invention generally relate to implantable systems and devices, and methods for use therewith, that can be used to perform arrhythmia discrimination. A plurality of different sensing vectors are used to obtain a plurality of different IEGMs, each of which is indicative of cardiac electrical activity at a different ventricular region. The plurality of different IEGMs can include, e.g., an IEGM indicative of cardiac electrical activity at a first region of the patient's LV chamber and an IEGM indicative of cardiac electrical activity at a second region of the patient's LV chamber. Additionally, the plurality of different IEGMs can further include an IEGM indicative of cardiac electrical activity at a region of a patient's RV chamber. For each of the IEGMs, there is a determination of a corresponding localized R-R interval stability metric indicative of the R-R interval stability at the corresponding ventricular region. This can include, e.g., determining, for each of the IEGMs, a plurality of R-R intervals corresponding to a plurality of consecutive cardiac cycles of the IEGM. For each IEGM, a measure of variation (e.g., standard deviation, range or variance, but not limited thereto) can then be determined for the plurality of R-R intervals to thereby determine the localized R-R interval stability metric for the IEGM. Arrhythmia discrimination is then performed using the plurality of determined R-R interval stability metrics.

In a specific embodiment, for each of the localized R-R interval stability metrics there is a determination of whether the localized R-R interval stability metric meets a stability criterion. This can include, e.g., comparing the localized R-R interval stability metric to an R-R interval stability threshold, and determining that the localized R-R interval stability metric meets the stability criterion if the localized R-R interval stability metric does not exceed the R-R interval stability threshold. Otherwise, there is a determination that the localized R-R interval stability metric does not meet the stability criterion.

If all of the localized R-R interval stability metrics meet the stability criterion, then the localized R-R interval stability metrics are indicative of a monomorphic tachycardia, e.g., monomorphic VT. If at least one of the localized R-R interval stability metrics meet the stability criterion while another one of the localized R-R interval stability metrics does not meet the interval stability criterion, then the localized R-R interval stability metrics are indicative of a tachycardia (e.g., VT) with intermittent functional block. If all of the localized R-R interval stability metrics do not meet the stability criterion, and all of the localized R-R interval stability metrics are within a similarity threshold of one another, then the localized R-R interval stability metrics are indicative of AF with fast irregular atrio-ventricular (AV) conduction, which is a type of SVT. If all of the localized R-R interval stability metrics do not meet the stability criterion, and all of the localized R-R interval stability metrics are not within the similarity threshold of one another, then the localized R-R interval stability metrics are indicative of polymorphic VT (PVT).

Further embodiments of the present invention are used to perform arrhythmia discrimination based on activation times. More specifically, a plurality of different sensing vectors are used to obtain a plurality of IEGMs that collectively enable electrical activations to be detected in the left atrial (LA) chamber, the right atrial (RA) chamber, and at least one ventricular chamber of a patient's heart. For each of a plurality of cardiac cycles, there is a determination, based on the plurality of obtained IEGMs, of an LA activation time, an RA activation time, and a ventricular activation time. Arrhythmia discrimination is then performed based on the determined activation times.

In specific embodiments, for each of the plurality of cardiac cycles there is a determination of an inter-atrial delay based on the LA and RA activation times. Further, there is a determination of whether the inter-atrial delays meet a uniformity criterion. If the inter-atrial delays do not meet the uniformity criterion, then it is determined that the inter-atrial delays are indicative of AF. If the inter-atrial delays meet the uniformity criterion, and at least a specified amount (e.g., all, N out of M, or X %) of the LA activation times occur before a corresponding ventricular activation time (or at least a predetermined delay before a corresponding ventricular activation time), then the activation times are indicative of an SVT with a rapid ventricular response. If the inter-atrial delays meet the uniformity criterion, and at least a specified amount (e.g., all, N out of M, or X %) of the LA activation times occur after a corresponding ventricular activation time (or at least a predetermined delay after a corresponding ventricular activation time), then the activation times are indicative of AVRT. If the inter-atrial delays meet the uniformity criterion, and at least a specified amount (e.g., all, N out of M, or X %) of the LA activation times occur at substantially the same time as (e.g., within a specified tolerance of) a corresponding ventricular activation time, then the activation times are indicative of AVNRT.

This summary is not intended to be a complete description of, or limit the scope of, the invention. Alternative and additional features, aspects, and objects of the invention can be obtained from a review of the specification, the figures, and the claims.

### BRIEF DESCRIPTION OF THE DRAWINGS

FIG. 1A is a simplified, partly cutaway view illustrating .an implantable stimulation device in electrical communication with at least three leads implanted into a patient's heart for delivering multi-chamber stimulation and shock therapy and sensing cardiac activity.

FIG. 1B is a functional block diagram of the multi-chamber implantable stimulation device of FIG. 1A, illustrating the basic elements that provide pacing stimulation, cardioversion, and defibrillation in four chambers of the heart.

FIG. 2 is a high level flow diagram that is used to describe techniques to monitor a patient's HF condition by monitoring localized cardiac function at a plurality of different regions of the patient's LV chamber and to select LV pacing sites for CRT therapy, according to embodiments of the present invention.

FIG. 3 illustrates an exemplary evoked response to a pacing pulse and exemplary features of the evoked response.

FIG. 4 is a high level flow diagram that is used to describe techniques to increase compliance with a predetermined ventricular electrical activation pattern, according to embodiments of the present invention.

FIG. 5 illustrates how an actual ventricular electrical activation pattern can be determined by detecting ventricular activations from a plurality of IEGMs obtained using a plurality of different sensing vectors corresponding to different ventricular regions, according to embodiments of the present invention.

FIG. 6 is a high level flow diagram that is used to describe techniques to perform arrhythmia discrimination based on a plurality of R-R interval stability metrics corresponding to a plurality of different ventricular regions, according to embodiments of the present invention.

FIG. 7 is a high level flow diagram that is used to provide additional details of one of the steps in FIG. 6, according to certain embodiments of the present invention.

FIG. 8 is a high level flow diagram that is used to describe techniques to perform arrhythmia discrimination based on activation times determined from a plurality of IEGMs obtained using a plurality of sensing vectors, according to embodiments of the present invention.

FIG. 9 is a high level flow diagram that is used to provide additional details of one of the steps in FIG. 8, according to certain embodiments of the present invention.

### DETAILED DESCRIPTION OF THE PREFERRED EMBODIMENTS

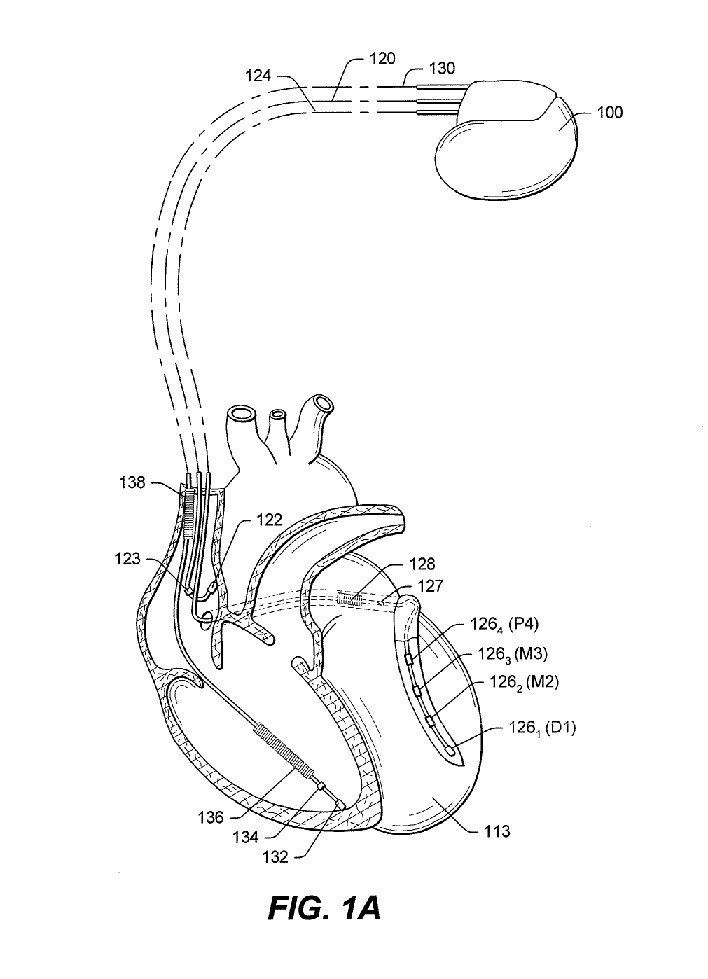

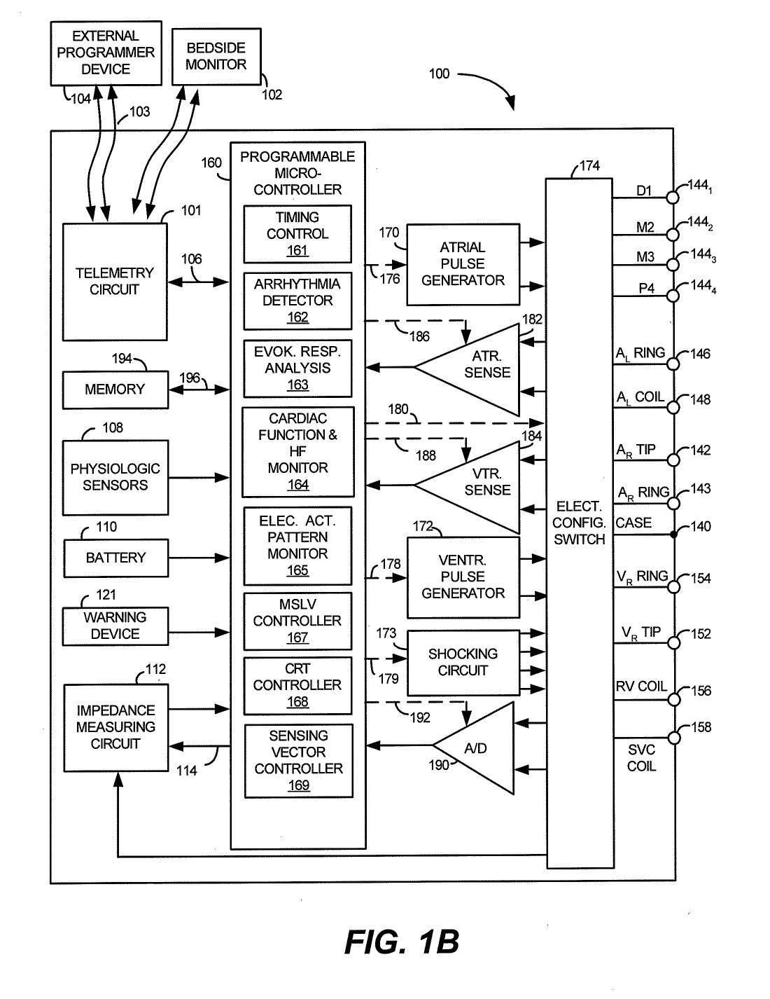

Embodiments of the present invention generally relate to chronically implantable cardiac monitoring and stimulation devices and systems such as pacemakers and/or implantable cardioverter-defibrillators (ICDs) and methods for use therewith. In particular, embodiments of the present invention can be used to monitor and treat heart failure (HF), increase compliance with a predetermined preferred ventricular electrical activation pattern, and/or perform arrhythmia discrimination. While not all of the embodiments are limited thereto, such embodiments are especially useful with implantable devices and systems capable of multi-site left ventricular (MSLV) pacing. In view of the above, an exemplary implantable cardiac system capable of delivering MSLV pacing, in which embodiments of the present invention described herein could be implemented, will now be described in conjunction with FIGS. 1A and 1B.

### Exemplary Pacemaker/ICD

With reference to FIGS. 1A and 1B, a description of an exemplary pacemaker/ICD will now be provided. FIG. 1A provides a simplified block diagram of the pacemaker/ICD, which is a dual-chamber stimulation device 100 capable of treating both fast and slow arrhythmias with stimulation therapy, including cardioversion, defibrillation, and pacing stimulation, including MSLV pacing. To provide atrial chamber pacing stimulation and sensing, pacemaker/ICD 100 is shown in electrical communication with a heart 113 by way of a right atrial (RA) lead 120 having an atrial tip electrode 122 and an atrial ring electrode 123 implanted in the atrial appendage. Pacemaker/ICD 100 is also in electrical communication with the heart by way of a right ventricular (RV) lead 130 having, in this embodiment, a ventricular tip electrode 132, a RV ring electrode 134, a RV coil electrode 136, and a superior vena cava (SVC) coil electrode 138. Typically, the RV lead 130 is transvenously inserted into the heart so as to place the RV coil electrode 136 in the RV apex, and the SVC coil electrode 138 in the superior vena cava. Accordingly, the RV lead is capable of receiving cardiac signals, and delivering stimulation in the form of pacing and shock therapy to the right ventricle (also referred to as the RV chamber).

To sense left atrial and ventricular cardiac signals and to provide left chamber pacing therapy, pacemaker/ICD 100 is coupled to a multi-pole LV lead 124 designed for placement in the “CS region” via the CS os for positioning a distal electrode adjacent to the left ventricle and/or additional electrode(s) adjacent to the left atrium (also referred to as the LA chamber). As used herein, the phrase “CS region” refers to the venous vasculature of the left ventricle, including any portion of the CS, great cardiac vein, left marginal vein, left posterior ventricular vein, middle cardiac vein, and/or small cardiac vein or any other cardiac vein accessible by the CS. Accordingly, an exemplary LV lead 124 is designed to receive atrial and ventricular cardiac signals and to deliver left ventricular pacing therapy using a set of four LV electrodes 126₁, 126₂, 126₃, and 126₄ (thereby providing a quadra-pole lead), left atrial pacing therapy using at least a LA ring electrode 127, and shocking therapy using at least a LA coil electrode 128. In certain embodiments, the LV lead 124 includes the LV electrodes 126₁, 126₂, 126₃, and 126₄, but does not include the LA electrodes 127 and 128. Such a lead can be, e.g., the Quartet™ left ventricular pacing lead developed by St. Jude Medical Inc. (headquartered in St. Paul, Minn.), which includes four pacing electrodes on the left ventricular lead—enabling up to 10 pacing configurations.

The LV electrode 126₁ is shown as being the most “distal” LV electrode (with relation to how far the electrode is from where the LV lead 124 connects to the pacemaker/ICD 100). The LV electrode 126₄ is shown as being the most “proximal” LV electrode. The LV electrodes 126₂ and 126₃ are shown as being “middle” LV electrodes, between the distal and proximal LV electrodes 126₁ and 126₄. Accordingly, so as to more aptly describe their relative locations, the four LV electrodes 126₁, 126₂, 126₃, and 126₄ can be referred to respectively as electrodes D1, M2, M3 and P4 (where “D” stands for “distal”, “M” stands for “middle”, and “P” stands from “proximal”, and the numbers are arranged from most distal to most proximal).

It is also possible that more or fewer LV electrodes are provided. However, for much of the remaining discussion, it will be assumed that the multi-pole LV lead 124 includes the four LV electrodes 126₁, 126₂, 126₃, and 126₄ (i.e., LV electrodes D1, M2, M3 and P4, respectively).

The four LV electrodes can be used to provide various different pacing vectors and sensing vectors. Some of the vectors are intraventricular LV vectors (vectors between two LV electrodes); whereas others are interventricular vectors (e.g., vectors between an LV electrode and the RV coil 136). Below is a list of exemplary vectors that can be used for pacing and/or sensing using the LV electrodes D1, M2, M3 and P4 with and without the RV coil 136. In the following list, the first electrode in each row (i.e., the electrode to the left of the arrow) is assumed to be connected as the cathode, and the second electrode in each row (i.e., the electrode to the right of the arrow) is assumed to be connected as the anode, but that need not be the case, especially where neither electrode is a coil.

D1→RV coil
M2→RV coil
M3→RV coil
P4→RV coil
D1→M2
D1→P4
M2→P4
M3→M2
M3→P4
P4→M2

Alternative and/or additional vectors, other than those listed above, can be used for pacing and/or sensing. Although only three leads are shown in FIG. 1A, it should also be understood that additional leads (with one or more pacing, sensing and/or shocking electrodes) might be used and/or additional electrodes might be provided on the leads already shown, such as additional electrodes on the RV or LV lead. It is also possible that less than three leads be used.

A simplified block diagram of internal components of pacemaker/ICD 100 is shown in FIG. 1B. While a particular pacemaker/ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation. The housing 140 for pacemaker/ICD 100, shown schematically in FIG. 1B, is often referred to as the “can”, “case” or “case electrode” and may be programmably selected to act as the return electrode for all “unipolar” modes. The housing 140 may further be used as a return electrode alone or in combination with one or more of the coil electrodes, 128, 136 and 138, for shocking purposes. The housing 140 further includes a connector (not shown) having a plurality of terminals, 142, 143, 144₁-144₄, 146, 148, 152, 154, 156 and 158 (shown schematically and, for convenience, the names of the electrodes to which they are connected are shown next to the terminals). As such, to achieve RA sensing and pacing, the connector includes at least a RA tip terminal (A_{R} TI P) 142 adapted for connection to the atrial tip electrode 122 and a RA ring (A_{R} RING) electrode 143 adapted for connection to RA ring electrode 123. To achieve left chamber sensing, pacing and shocking, the connector includes an LV tip terminal 144₁ adapted for connection to the D1 electrode and additional LV electrode terminals 144₂, 144₃ and 144₄ terminals adapted for connection to the M2, M3 and P4 electrodes of the quadra-pole LV lead.

The connector also includes a LA ring terminal (A_{L} RING) 146 and a LA shocking terminal (A_{L} COIL) 148, which are adapted for connection to the LA ring electrode 127 and the LA coil (A_{L} COIL) electrode 128, respectively. To support right chamber sensing, pacing and shocking, the connector further includes a RV tip terminal (V_{R} TIP) 142, a RV ring terminal (V_{R} RING) 143, a RV shocking terminal (V_{R} COIL) 156, and an SVC shocking terminal (SVC COIL) 158, which are adapted for connection to the RV tip electrode 132, RV ring electrode 134, the RV coil electrode 136, and the SVC coil electrode 138, respectively.

At the core of pacemaker/ICD 100 is a programmable microcontroller 160, which controls the various modes of stimulation therapy. As is well known in the art, the microcontroller 160 (also referred to herein as a control unit or controller) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 160 includes the ability to process or monitor input signals (data) as controlled by a program code stored in a designated block of memory. The details of the design and operation of the microcontroller 160 are not critical to the invention. Rather, any suitable microcontroller 160 may be used that carries out the functions described herein. The use of microprocessor-based control circuits for performing timing and data analysis functions are well known in the art.

As shown in FIG. 1B, an atrial pulse generator 170 and a ventricular pulse generator 172 generate pacing stimulation pulses for delivery by the RA lead 120, the RV lead 130, and/or the LV lead 124 via an electrode configuration switch 174. It is understood that in order to provide stimulation therapy in each of the four chambers of the heart, the atrial and ventricular pulse generators, 170 and 172, may include dedicated, independent pulse generators, multiplexed pulse generators or shared pulse generators. The pulse generators, 170 and 172, are controlled by the microcontroller 160 via appropriate control signals, 176 and 178, respectively, to trigger or inhibit the stimulation pulses.

The microcontroller 160 includes timing control circuitry 161 to control the timing of the stimulation pulses, including, but not limited to, pacing rate, atrio-ventricular (AV) delay, interatrial conduction (AA) delay, interventricular conduction (VV) delay and/or intraventricular delay (e.g., LV1-LV2 delay). The timing control circuitry 161 can also keep track of the timing of refractory periods, blanking intervals, noise detection windows, evoked response detection windows, alert intervals, marker channel timing, etc., which is well known in the art.

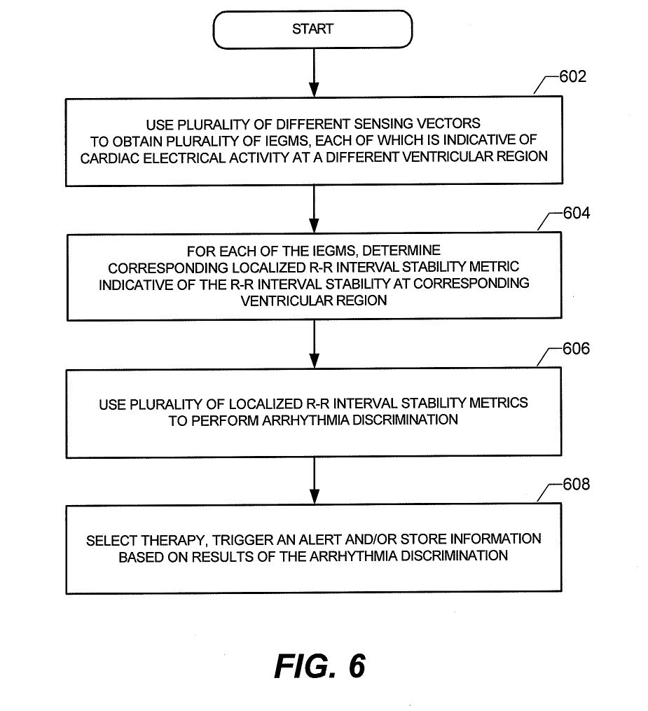

The microcontroller 160 further includes an arrhythmia detector 162. The detector 162 can be utilized by the stimulation device 100 for determining desirable times to administer various therapies. The arrhythmia detector 162 can perform various arrhythmia discrimination techniques, some of which are described herein with reference to FIGS. 6-9, so that appropriate therapy can be selectively provided to the patient. The detector 162 may be implemented in hardware as part of the microcontroller 160, or as software/firmware instructions programmed into the device and executed on the microcontroller 160 during certain modes of operation. The arrhythmia detector can also initiate the saving of information regarding arrhythmias, including, but not limited, information about characterizations of arrhythmias, IEGM information corresponding to periods of time during which arrhythmias are detected, therapies delivered in response to detection and/or diagnosis of arrhythmia, and the electrical and physiologic responses to such therapies.

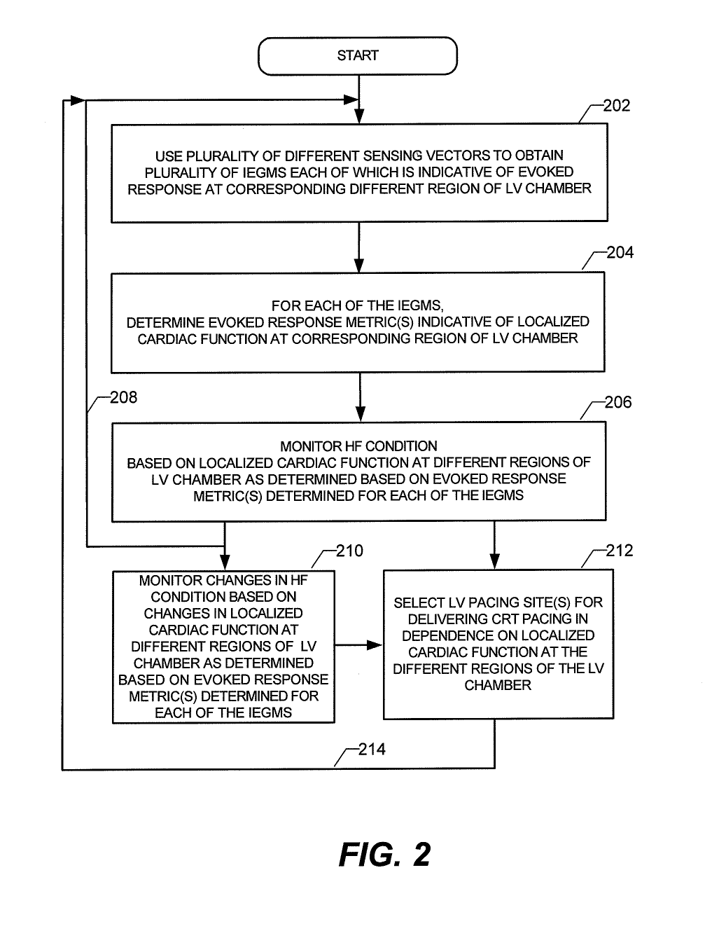

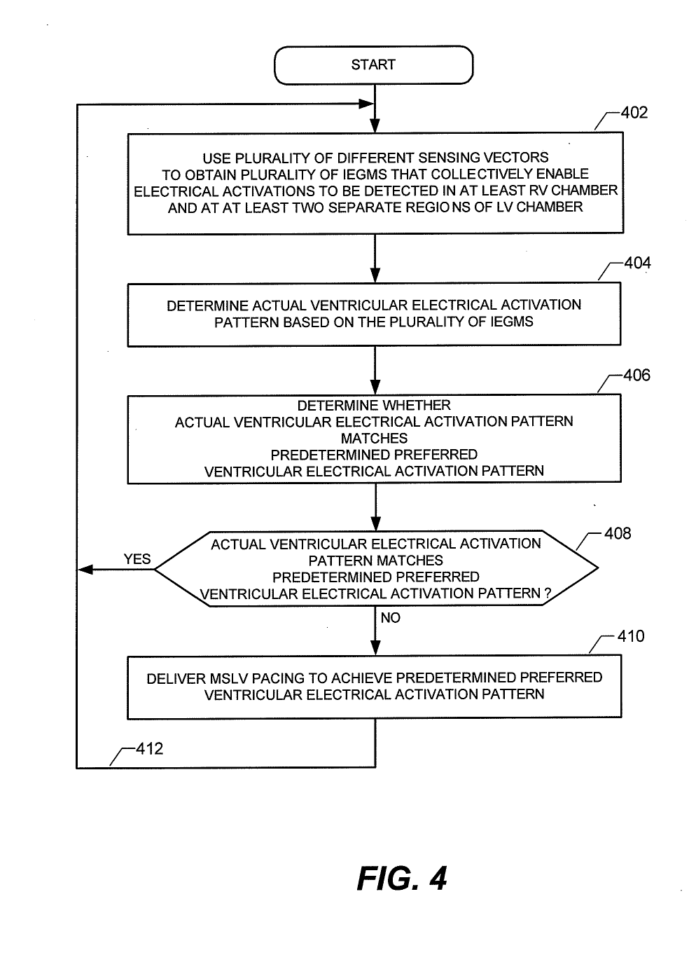

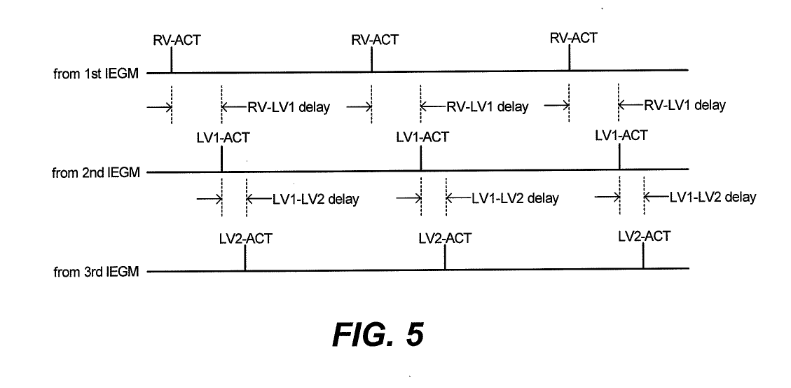

The microcontroller 160 further includes an evoked response analysis module 163, a cardiac function and HF monitor module 164, and an electrical activation pattern monitor module 165. These modules can be used to implement various algorithms and/or methods presented below in the discussion of FIGS. 2-5. The aforementioned components may be implemented in hardware as part of the microcontroller 260, or as software/firmware instructions programmed into the device and executed on the microcontroller 160 during certain modes of operation. The evoked response analysis module 163, as described herein, may aid in the acquisition, analysis, etc., of information related to IEGMs and, in particular, analyzing evoked responses to pacing pulses in accordance with embodiments of the present invention. The cardiac function and HF monitor module 164 may aid in the monitoring of localized cardiac function and the monitoring of HF. The electrical activation pattern monitor module 165 can aid in monitoring a patient's actual electrical activation pattern. Additionally, the electrical activation monitor module 165 can be used to determine whether a patient's actual electrical activation pattern matches a predetermined preferred electrical activation pattern, and to selectively trigger MSLV pacing when there is not a match, as described below with reference to FIGS. 4 and 5.

Additional components of the microcontroller include a MSLV controller 167 to control the actual delivery of MSLV pacing and a CRT controller 168 to control CRT, which can be performed in conjunction with MSLV pacing.

The microcontroller 160 is also shown as including a sensing vector controller 169 that can be used, e.g., to control the electrode configuration switch 174 (e.g., via control signals 180) to selectively connect specific electrode(s) to the sensing circuits 182 or 184 as a cathode or an anode, to achieve the various sensing vectors that are used to obtain IEGMs in accordance with embodiments of the present invention. Where multiple sensing vectors are being used to obtain a plurality of IEGMs indicative of cardiac electrical activity at a plurality of ventricular regions, the sensing circuit 184 may include multiple channels (e.g., duplicate circuitry) to enable sensing of more than one ventricular IEGM signal at the same time, and/or the sensing circuit 184 may use time divisional multiplexing to sense more than one ventricular IEGM signal.

Depending upon the implementation, the various components of the microcontroller may be implemented as separate software modules or the modules may be combined to permit a single module to perform multiple functions. For example, the MSLV controller and the CRT controller 168 can be combined. In addition, although shown as being components of the microcontroller, some or all of these components may be implemented separately from the microcontroller, using application specific integrated circuits (ASICs) or the like.

Switch 174 includes a plurality of switches for connecting the desired electrodes to the appropriate I/O circuits, thereby providing complete electrode programmability. Accordingly, the switch 174, in response to a control signal 180 from the microcontroller 160, determines the polarity of the stimulation pulses (e.g., unipolar, bipolar, combipolar, etc.) by selectively closing the appropriate combination of switches (not shown) as is known in the art. The switch also switches among the various LV electrodes.

Atrial sensing circuits 182 and ventricular sensing circuits 184 may also be selectively coupled to the RA lead 120, LV lead 124, and the RV lead 130, through the switch 174 for detecting the presence of cardiac activity in each of the four chambers of the heart. Accordingly, the atrial (ATR. SENSE) and ventricular (VTR. SENSE) sensing circuits, 182 and 184, may include dedicated sense amplifiers, multiplexed amplifiers or shared amplifiers. The switch 174 determines the “sensing polarity” of the cardiac signal by selectively closing the appropriate switches, as is also known in the art. In this way, the clinician may program the sensing polarity independent of the stimulation polarity. Each sensing circuit, 182 and 184, preferably employs one or more low power, precision amplifiers with programmable gain and/or automatic gain control, bandpass filtering, and a threshold detection circuit, as known in the art, to selectively sense the cardiac signal of interest. The automatic gain control enables pacemaker/ICD 100 to deal effectively with the difficult problem of sensing the low amplitude signal characteristics of atrial or ventricular fibrillation. The outputs of the atrial and ventricular sensing circuits, 182 and 184, are connected to the microcontroller 160 which, in turn, are able to trigger or inhibit the atrial and ventricular pulse generators, 170 and 172, respectively, in a demand fashion in response to the absence or presence of cardiac activity in the appropriate chambers of the heart.

For arrhythmia detection, pacemaker/ICD 100 utilizes the atrial and ventricular sensing circuits, 182 and 184, to sense cardiac signals to determine whether a rhythm is physiologic or pathologic. As used in this section “sensing” is reserved for the noting of an electrical signal, and “detection” is the processing of these sensed signals and noting the presence of an arrhythmia, an evoked response, an intrinsic event, or some other event being monitored for. The timing intervals between sensed events (e.g., AS, VS, and depolarization signals associated with fibrillation which are sometimes referred to as “F-waves” or “Fib-waves”) can be classified by the microcontroller 160 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks). The arrhythmia detector 162, mentioned above, can be used to detect and characterize such arrhythmias, e.g., using embodiments of the present invention described with reference to FIGS. 6-9, but not limited thereto.

Cardiac signals are also applied to the inputs of an analog-to-digital (A/D) data acquisition system 190. The data acquisition system 190 is configured to acquire intracardiac electrogram signals, convert the raw analog data into a digital signal, and store the digital signals for later processing and/or telemetric transmission to an external programmer 104 or a bedside monitor or personal advisory module (PAM) 102. The data acquisition system 190 is coupled to the RA lead 120, the LV lead 124, and the RV lead 130 through the switch 174 to sample cardiac signals across any pair of desired electrodes. The microcontroller 160 is further coupled to a memory 194 by a suitable data/address bus 196, wherein the programmable operating parameters used by the microcontroller 160 are stored and modified, as required, in order to customize the operation of pacemaker/ICD 100 to suit the needs of a particular patient. Such operating parameters define, for example, the amplitude or magnitude, pulse duration, electrode polarity, for both pacing pulses and impedance detection pulses as well as pacing rate, sensitivity, arrhythmia detection criteria, and the amplitude, waveshape and vector of each pacing and shocking pulse to be delivered to the patient's heart within each respective tier of therapy. Other pacing parameters include base rate, rest rate and circadian base rate.

Advantageously, the operating parameters of the implantable pacemaker/ICD 100 may be non-invasively programmed into the memory 194 through a telemetry circuit 101 in telemetric communication with an external device 104 or bedside monitor 102, such as a programmer, transtelephonic transceiver or a diagnostic system analyzer. The telemetry circuit 101 is activated by the microcontroller by a control signal 106. The telemetry circuit 101 advantageously allows intracardiac electrograms and status information relating to the operation of pacemaker/ICD 100 (as contained in the microcontroller 160 or memory 194) to be sent to the external device 102 through an established communication link 103. An internal warning device 121 (also referred to as a patient alert) may be provided for generating perceptible warning signals to the patient via vibration, voltage or other methods.

Pacemaker/ICD 100 further includes an accelerometer or other physiologic sensor 108, commonly referred to as a “rate-responsive” sensor because it is typically used to adjust pacing stimulation rate according to the exercise state of the patient. However, the physiological sensor 108 may further be used to detect changes in cardiac output, changes in the physiological condition of the heart, or diurnal changes in activity (e.g., detecting sleep and wake states) and to detect arousal from sleep. Accordingly, the microcontroller 160 can respond by adjusting the various pacing parameters (such as rate, AV delay, W delay, etc.) at which the atrial and ventricular pulse generators, 170 and 172, generate stimulation pulses. While shown as being included within pacemaker/ICD 100, it is to be understood that the physiologic sensor 108 may also be external to pacemaker/ICD 100, yet still be implanted within or carried by the patient. A common type of rate responsive sensor is an activity sensor incorporating an accelerometer or a piezoelectric crystal, which is mounted within the housing 140 of pacemaker/ICD 100. Other types of physiologic sensors are also known, for example, sensors that sense the oxygen content of blood, respiration rate and/or minute ventilation, pH of blood, ventricular gradient, stroke volume, cardiac output, contractility, etc.

The pacemaker/ICD additionally includes a battery 110 , which provides operating power to all of the circuits shown in FIG. 1B. The battery 110 may vary depending on the capabilities of pacemaker/ICD 100. If the system only provides low voltage therapy, a lithium iodine or lithium copper fluoride cell typically may be utilized. For pacemaker/ICD 100, which employs shocking therapy, the battery 110 should be capable of operating at low current drains for long periods, and then be capable of providing high-current pulses (for capacitor charging) when the patient requires a shock pulse. The battery 110 should also have a predictable discharge characteristic so that elective replacement time can be detected. Accordingly, appropriate batteries are employed.

As further shown in FIG. 1B, pacemaker/ICD 100 is shown as having an impedance measuring circuit 112, which is enabled by the microcontroller 160 via a control signal 114. Uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; and detecting the opening of heart valves, etc. The impedance measuring circuit 112 is advantageously coupled to the switch 174 so that any desired electrode may be used.

In the case where pacemaker/ICD 100 is intended to operate as an implantable cardioverter/defibrillator (ICD) device, it detects the occurrence of an arrhythmia, and automatically applies an appropriate electrical shock therapy to the heart aimed at terminating the detected arrhythmia. To this end, the microcontroller 160 further controls a shocking circuit 173 by way of a control signal 179. The shocking circuit 173 generates shocking pulses of low (up to 0.1 joules), moderate (0.1-10 joules) or high energy (11 to 40 joules or more), as controlled by the microcontroller 160. Such shocking pulses are applied to the heart of the patient through at least two shocking electrodes, and as shown in this embodiment, selected from the LA coil electrode 128, the RV coil electrode 136, and/or the SVC coil electrode 138. The housing 140 may act as an active electrode in combination with the RV electrode 136, or as part of a split electrical vector using the SVC coil electrode 138 or the LA coil electrode 128 (i.e., using the RV electrode as a common electrode). Cardioversion shocks are generally considered to be of low to moderate energy level (so as to minimize pain felt by the patient), and/or synchronized with a R-wave and/or pertaining to the treatment of tachycardia. Defibrillation shocks are generally of moderate to high energy level (i.e., corresponding to thresholds in the range of 7-40 joules), delivered asynchronously (since R-waves may be too disorganized), and pertaining exclusively to the treatment of fibrillation. Accordingly, the microcontroller 160 is capable of controlling the synchronous or asynchronous delivery of the shocking pulses.

The above described implantable device 100 was described as an exemplary pacemaker/ICD. One or ordinary skill in the art would understand that embodiments of the present invention can be used with alternative types of implantable devices. Accordingly, embodiments of the present invention should not be limited to use only with the above described device.

### Monitoring a Patient'S HF Condition Based on Localized Cardiac Function at the Plurality of Different Regions of the LV Chamber

FIG. 2 is a high level flow diagram that is used to describe techniques to monitor a patient's HF condition by monitoring localized cardiac function at a plurality of different regions of the patient's LV chamber, according to embodiments of the present invention. Additionally, the techniques described with reference to FIG. 2 can be used select one or more LV pacing sites for CRT therapy.

Referring to FIG. 2, at step 202 a plurality of different sensing vectors are used to obtain a plurality of intracardiac electrograms (IEGMs) each of which is indicative of an evoked response at a corresponding different region of the LV chamber. Two or more IEGMs are obtained at step 202. Preferably, these IEGMs are obtained while pacing is being performed at at least one site within the LV chamber. Such pacing of the LV chamber can be part of Bi-V pacing, but need not be.

The sensing vectors used at step 202 can include, for example, a first sensing vector having a cathode (e.g., the D1 electrode 126₁) at a first LV (LV1) site within the LV chamber, which can be used to obtain a first IEGM that enables evoked responses to be detected at the LV1 site. Further, a second sensing vector having a cathode (e.g., the P4 electrode 126₄) at a second LV (LV2) site within the LV chamber can be used to obtain a second IEGM that enables evoked responses to be detected at the LV2 site. One or more additional sensing vectors can also be used to detect the evoked responses at one or more further regions of the LV chamber, to thereby obtain information about the localized cardiac function at the one or more further regions of the LV chamber. For each of the above exemplary sensing vectors, the anode electrode can be the RV coil electrode 136, the case electrode 140, or the SVC coil electrode 138, but is not limited thereto. These are just exemplary sensing vectors that can be used at step 202, which are not meant to be limiting.

At step 204, for each of the IEGMs, there is a determination of one or more evoked response metrics indicative of the cardiac function at the corresponding region of the LV chamber. In accordance with an embodiment, the evoked response metrics obtained at step 204 are the paced depolarization integral (PDI). Alternatively, or additionally, the evoked response metrics obtained at step 204 can be the maximum upward slope of an R-wave.

Preferably each time steps 202 and 204 are performed, ventricular evoked response metric(s) is/are determined for each of a plurality of cardiac cycles, and metrics of the same type (e.g., PDI) are combined, e.g., averaged, summed, filtered (according to signal stability and/or quality), heart rate corrected, or the like, to reduce the affects of noise and motion artifacts on such measurements. For example, the PDI for 60 cardiac cycles of an IEGM can be determined and averaged to produce the PDI evoked response metric that is indicative of localized cardiac function. Additionally, or alternatively, the maximum upward slope of an R-wave for the same 60 cardiac cycles can be measured and averaged to produce the maximum upward slope evoked response metric.

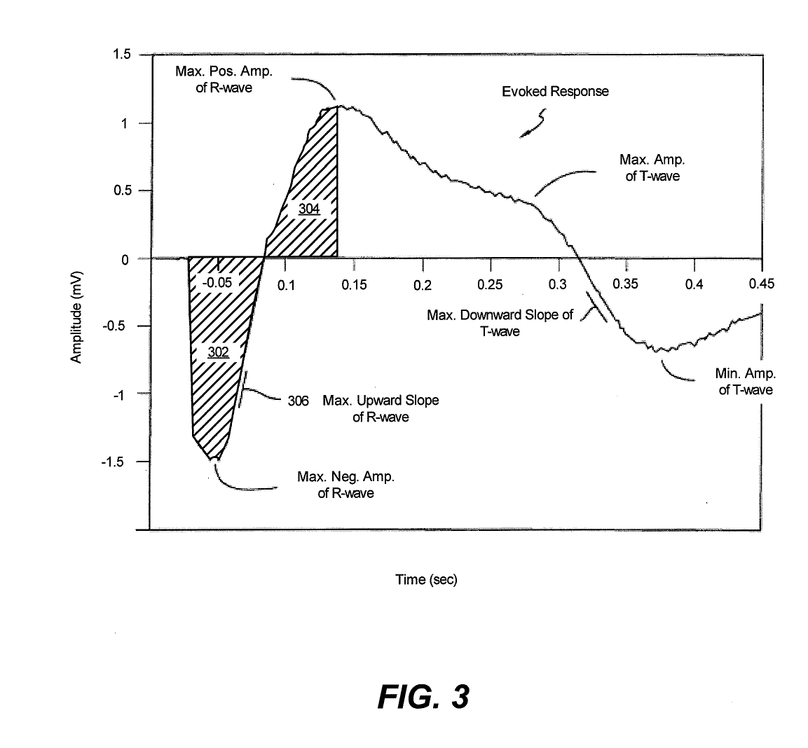

Referring briefly to FIG. 3, depending upon implementation, the PDI can corresponding to the area 302 of the evoked response waveform, the area 304 of the evoked response waveform, or a sum the areas 302 and 304. Still referring to FIG. 3, reference number 306 illustrates that maximum upward slope of an R-wave of the evoked response waveform.

A low PDI and a low maximum upward slope of an R-wave are indicative of a low localized cardiac function. Conversely, a high PDI and a high maximum upward slope of an R-wave are indicative of a high localized cardiac function. Accordingly, decreases in PDI and decreases in the maximum upward slope of an R-wave are indicative of a reduction in localized cardiac function, whereas increases in the same are indicative of increases in localized cardiac function. Localized low cardiac function (e.g., observed as low evoked response metrics) can be indicative of local myocardial remodeling and/or stretching, which is/are preferably mitigated.

Alternative and/or additional evoked response metrics that are believed to be indicative of localized cardiac function, include, but are not limited to, maximum positive R-wave amplitude and maximum negative R-wave amplitude. A low maximum positive amplitude and low maximum negative amplitude are believed to be indicative of a low localized cardiac function. Conversely, a high maximum positive amplitude and a high maximum negative amplitude of an R-wave are believed to be indicative of a high localized cardiac function. Accordingly, decreases in such amplitudes are indicative of a reduction in localized cardiac function, whereas increases in the same are indicative of increases in localized cardiac function. Uses of alternative and/or additional evoked response metrics (e.g., extent of fractionation) are also possible.

Returning to FIG. 2, at step 206, the patient's HF condition is monitored based on the localized cardiac function at the plurality of different regions of the LV chamber as determined based on the one or more evoked response metrics determined from each of the IEGMs. More generally, the patient's HF condition is monitored based on the evoked response metrics associated with a plurality of different regions of the LV chamber. If the patient has a relatively high localized cardiac function (and more generally, relatively high evoked response metrics) at all of the regions of the LV chamber for which evoked response metrics are determined, then the patient's HF condition can be considered to be good or under control. If the patient has a relatively low localized cardiac function (and more generally, relatively low evoked response metrics) at all of the regions of the LV chamber for which evoked response are determined, then the patient's HF condition can be considered to be poor. If the patient has a relatively moderate and similar localized cardiac function (and more generally, relatively moderate and similar evoked response metrics) at all of the regions of the LV chamber for which evoked response are determined, then the patient's HF condition can be considered to be moderate, and the LV electrical substrate can be considered to be relatively uniform or homogeneous. If the patient has a relatively low localized cardiac function at at least one of the regions of the LV chamber for which evoked responses are determined while having relatively moderate or high localized cardiac function at at least another one of the regions of the LV chamber for which evoked responses are determined, then the patient's HF condition can also be considered to be between low and moderate, and the LV electrical substrate can be considered to be non-uniform or heterogeneous. Other variations are also possible, and within the scope of the present invention.

Evoked response metric thresholds can be programmed into an implantable device and used to characterize localized cardiac function and/or HF status. Such thresholds can be patient specific and/or based on a patient population. It is also possible that baseline thresholds be determined at a point in time (e.g., soon after implantation of an implantable system), and that the baseline thresholds be used to determine whether localized cardiac function and/or overall HF condition is/are improving, worsening or staying relatively the same. Alternative or additional HF classifiers (other than low, high and moderate) can also be used. It is also possible that an algorithm, look-up table, or the like, can be used to determine the patient's HF condition based on the various evoke response metrics determined at step 204. In certain embodiments, the patient's HF condition can be quantized by averaging, adding or otherwise combining values that are indicative of the localized cardiac function at the plurality of different regions of the LV chamber. Further, as part of the HF monitoring, embodiments of the present invention can be used to analyze the uniformity (also referred to as homogeneity) of the electrical substrate of the LV chamber. In general, it is believed that it is better for the LV chamber to have a homogeneous electrical substrate than a heterogeneous electrical substrate.

As indicated by line 208, steps 202, 204 and 206 can be repeated from time to time, to thereby enable changes in the patient's HF condition to be monitored at step 210 based on changes in the localized cardiac function at one or more of the plurality of different regions of the LV chamber as determined based on the one or more evoked response metrics determined from each of the IEGMs. For example, these steps can be continually repeated, repeated every minute, repeated every hour, or repeated at some other time interval. It is also possible that there is a triggering event that causes these steps to be performed. By monitoring for changes in the patient's HF condition, prediction or early detection of an HF exacerbation can be achieved.

As indicated by step 212, one or more LV pacing sites can be selected for delivering cardiac resynchronization therapy (CRT) pacing in dependence on the localized cardiac function at the plurality of different regions of the patient's LV chamber. In specific embodiments, step 212 involves selecting one or more LV pacing sites that improve the patient's HF condition. This can be accomplished by attempting to increase (and potentially maximize) the localized cardiac function at the various LV regions. More generally, such increases can be recognized by increases in the evoked response metrics. For example, in certain embodiments, one or more LV pacing sites can be selected in an attempt to increase the PDI and/or increase the maximum upward slope of an R-wave.

In accordance with an embodiment, step 212 involves selecting one or more LV pacing sites that increase a similarity between evoked response metrics indicative of the localized cardiac function at a first region of the LV chamber (e.g., corresponding to an LV1 site) and corresponding evoked response metrics indicative of the localized cardiac function at a second region of the LV chamber (e.g., corresponding to an LV2 site). Increasing the similarity of corresponding evoked response metrics at different LV regions increases the uniformity (also referred to as homogeneity) of the LV electrical substrate, which is believed to be advantageous. This concept of attempting to increase similarity between evoked response metrics indicative of the localized cardiac function can be extended to more than two LV sites, e.g., also an LV3 site, and potentially an LV4 site.

In accordance with an embodiment, step 212 involves selecting, as an LV pacing site for delivering CRT pacing, the region within the LV chamber that is identified, based on evoked response metrics, to have the lowest localized cardiac function. It may be beneficial to select the site having a lowest localized cardiac function (and more generally, the lowest evoked response metric) as the pacing site to thereby improve the localized cardiac function at that site, so long as it is viable. An evoked response at a viable site should change with changing pacing parameters, and have at least a minimum PDI and a minimum maximum upward slope of an R-wave. Thus, this embodiment can also include determining whether a particular region is viable before selecting that region as a pacing site. This may be advantageous, since pacing at a non-viable site may be detrimental. More specifically non-viable (e.g., scar) site can be act as an arrhythmogenic substrate, and repetitive pacing in the immediate vicinity of the non-viable site can potentially trigger VTs. Viability can be tested at a site by varying a pacing parameter (e.g., pulse amplitude) used to pace at that site, and determining whether the evoked response changes with the change to the pacing parameter, but is not limited thereto.

In accordance with another embodiment, step 212 involves selecting, as an LV pacing site for delivering CRT pacing, the region within the LV chamber that is identified, based on evoked response metrics, to have the highest localized cardiac function. It may be beneficial to select the site having a highest localized cardiac function (and more generally, the highest evoked response metric) as the pacing site since the site having a highest localized cardiac function may be more responsive to pacing and therefore confer the largest increase to the global cardiac function.

In certain embodiments, evoked response metrics are determined for up to four different LV regions, e.g., using each of the D1, M2, M3 and P4 electrodes (126₁-126₄) individually as the cathodes of four different sensing vectors. In such embodiments, step 212 can involve selecting at least two LV sites for multi-site LV (MSLV) pacing. For example, the two sites having the lowest localized cardiac function may be paced. For another example, the two sites having the highest localized cardiac function may be paced. In still another example, the two sites having the highest and lowest localized cardiac function may be paced. In a further example, the two sites having neither the highest nor the lowest localized cardiac function may be paced.

In still other embodiments, a predetermined one of the D1, M2, M3 and P4 electrodes can always be used for Bi-V pacing, and at step 212 an additional one of the D1, M2, M3 and P4 electrodes can be selected to achieve MSLV pacing. Additionally, different intra-LV delays can be tested in order to select a delay that provides increased localized cardiac function at the various LV regions and/or increased uniformity of the LV electrical substrate.

As can be appreciated from line 214, as the localized cardiac function at the various LV regions changes, and thus the patient's HF condition changes, the LV pacing site(s) selected at step 212 can change.

Additionally, an alert (e.g., warning device 121) can be selectively triggered based on the patient's HF condition, or a change therein. In an embodiment, the alert can be triggered based on the overall HF condition being monitored, e.g., if it falls below a threshold, or if a change in the patient's HF condition indicates worsening beyond a corresponding change threshold. Alternatively, or additionally, an alert can be triggered in response to one or more of the evoked response metrics, or a combination thereof, falling below a corresponding threshold. It is also possible that an alert can be triggered to notify medical personal of the patient's HF condition, or a change therein, reaches a corresponding threshold.

Also, information indicative of the patient's HF condition can be saved. This can include saving information about the overall HF condition of the patient, saving evoked response metric information, localized cardiac function information, and/or saving portions of IEGMs from which the patient's HF condition is assessed.

In accordance with an embodiment, multiple sensing vectors can be ganged together to provide a global sense channel. Such a global sense channel can be used to determine an overall HF condition of patient's LV chamber. Analysis of the global sense channel can be used, e.g., prior to one of the embodiments described with reference to the flow diagram of FIG. 2, e.g., to trigger determining more localized LV cardiac function. Additionally, or alternatively, the global sense channel can be used after the one of embodiments described with reference to FIG. 2, to determine whether the overall HF condition is such that it is (or is not) worth adjusting CRT pacing. For example, if the patient's overall HF condition as determined using a global sense channel is at least moderate, it may not be worth adjusting the CRT pacing the is presently being used. If the patient's overall HF condition as determined using a global sense channel is below moderate, it may be worth adjusting the CRT pacing the to see if an improvement can be achieved.

Increasing Compliance with a Predetermined Preferred Ventricular Electrical Activation Pattern

A cardiologist or other physician may be determine that a patient has a predetermined preferred ventricular electrical activation pattern, e.g., for physiologic reasons, such as, but not limited, improving and preferably maximizing cardiac mechanical function. Such a predetermined preferred ventricular electrical activation pattern can include a predetermined preferred ventricular electrical activation sequence, and at least two predetermined preferred ventricular electrical activation delays. In accordance with an embodiment, the predetermined preferred ventricular electrical activation delays include one or more predetermined preferred intra-ventricular delay(s), e.g., an LV1-LV2 delay, and possibly an LV2-LV3 delay. Further intra-ventricular delays are also possible, e.g., an LV3-LV4 delay. Additionally, the predetermined preferred ventricular electrical activation delays can include an inter-ventricular delay, i.e., an RV-LV delay or an LV-RV delay.

For a specific example, the predetermined preferred ventricular electrical activation sequence can be an RV-LV1-LV2 sequence, and the predetermined preferred ventricular electrical activation delays can include a predetermined preferred RV-LV1 delay (or a range of predetermined preferred RV-LV1 delays) and a predetermined preferred LV1-LV2 delay (or a range of predetermined preferred LV1-LV2 delays). For another example, the predetermined preferred ventricular electrical activation sequence can be an LV1-LV2-RV sequence, and the predetermined preferred ventricular electrical activation delays can include a predetermined preferred LV1-LV2 delay (or a range of predetermined preferred LV1-LV2 delays) and a predetermined preferred LV2-RV delay (or a range of predetermined preferred LV2-RV delays). For still a further example, the predetermined preferred ventricular electrical activation sequence can be an RV-LV1-LV2-LV3 sequence, and the predetermined preferred ventricular electrical activation delays can include a predetermined preferred RV-LV1 delay (or a range of predetermined preferred RV-LV1 delays), a predetermined preferred LV1-LV2 delay (or a range of predetermined preferred LV1-LV2 delays), and a predetermined preferred LV2-LV3 delay (or a range of predetermined preferred LV2-LV3 delays). For yet another example, the predetermined preferred ventricular electrical activation sequence can be an LV1-RV-LV2 sequence, and the predetermined preferred ventricular electrical activation delays can include a predetermined preferred LV1-RV delay (or a range of predetermined preferred LV1-RV delays), a predetermined preferred RV-LV2 delay (or a range of predetermined preferred RV-LV2 delays). A range of predetermined preferred LV1-LV2 delays and a range of predetermined preferred LV1-RV delays may in some cases overlap.

For a further example, the predetermined preferred ventricular electrical activation sequence can indicate that an activation should first occur at a particular region (e.g., an LV1 region), and that activations at two or more other regions (e.g., an LV2 region and an RV region) occur after the activation at the first region (e.g., after the LV1 activation). Continuing with this example, the predetermined preferred ventricular electrical activation delays can include a predetermined preferred LV1-LV2 delay (or a range of predetermined preferred LV1-LV2 delays), and a predetermined preferred LV1-RV delay (or a range of predetermined preferred LV1-RV delays). In other words, in this example, more than one predetermined preferred electrical activation delay can specify a delay from a common activation (i.e., from the LV1 activation in this example). Again, a range of predetermined preferred LV1-LV2 delays and a range of predetermined preferred LV1-RV delays may in some cases overlap.

Other predetermined preferred ventricular electrical activation patterns are also possible. Ventricular depolarization causes ventricular activation, and thus, for the purpose of these embodiments those two terms can be used interchangeably and are considered to happen at substantially the same time.

In accordance with specific embodiments of the present invention described with reference to FIGS. 4 and 5, information defining a predetermined preferred ventricular electrical activation pattern can be programmed into an implantable stimulation device, and MSLV pacing can be selectively delivered to increase compliance with the predetermined preferred ventricular electrical activation pattern. Reference is first made to the high level flow diagram of FIG. 4. Referring to FIG. 4, at step 402 a plurality of different sensing vectors are used to obtain a plurality of IEGMs that collectively enable electrical activations to be detected in at least the RV chamber and at at least two separate regions of the LV chamber. For example, a sensing vector having a cathode (e.g., the RV tip electrode 132 or the RV ring electrode 134) within the RV chamber can be used to obtain an IEGM that enables electrical activations to be detected in the RV chamber. Additionally, a sensing vector having a cathode (e.g., the D1 electrode 126₁) at a first LV (LV1) site within the LV chamber can be used to obtain a further IEGM that enables electrical activations to be detected at the LV1 site. Further, a sensing vector having a cathode (e.g., the P4 electrode 126₄) at a second LV (LV2) site within the LV chamber can be used to obtain an IEGM that enables electrical activations to be detected at the LV2 site. For each of the above exemplary sensing vectors, the anode electrode can be the RV coil electrode 136, the case electrode 140 or the SVC coil electrode 138, but is not limited thereto. These are just exemplary sensing vectors that can be used to obtain a plurality of IEGMs at step 402, which are not meant to be limiting.

At step 404, an actual ventricular electrical activation pattern is determined based on the plurality of IEGMs obtained at step 402. The actual electrical activation pattern includes an actual ventricular electrical activation sequence, and at least two actual ventricular activation delays. For example, the actual electrical activation sequence can be the sequence LV1-LV2-RV, and the actual ventricular activation delays can be an actual LV1-LV2 delay, and an actual LV2-RV delay. For another example, the actual electrical activation sequence can be the sequence RV-LV1-LV2, and the actual ventricular activation delays can be an actual RV-LV1 delay, and an actual LV1-LV2 delay. For still another example, the actual electrical activation sequence can be the sequence LV1-RV-LV2, and the actual ventricular activation delays can be an actual LV1-RV delay, and an actual RV-LV2 delay. Alternatively, it can be that the actual RV and LV2 activations occur substantially simultaneously, after the actual LV activation, in which case there can be an actual LV1-RV delay that is substantially equal to an actual LV1-LV2 delay. These are just a few examples, which are not meant to be all inclusive.

At step 406, there is a determination of whether the actual ventricular electrical activation pattern matches the predetermined preferred ventricular electrical activation pattern. In accordance with an embodiment, step 406 includes determining whether the actual ventricular electrical activation sequence is the same as the predetermined preferred ventricular electrical activation sequence. If they are the same, then there is a determination of whether the actual delays match the predetermined preferred delays. Where each predetermined preferred delay is a specific delay (as opposed to a range of delays), then the actual ventricular electrical activation delays can be considered to match the predetermined preferred ventricular electrical activation delays where the actual delays are within a predetermined tolerance of the predetermined preferred delays. The predetermined tolerance can be programmed, and can be defined, e.g., by a percentage (e.g., 10%) or by an absolute difference (e.g., 10 ms), but is not limited thereto. Where predetermined preferred delays are specified as a range of delays, the actual ventricular electrical activation delays can be considered to match the predetermined preferred ventricular electrical activation delays where the actual delays are within the specified range of delays.

The determination of whether or not there is a match (between the actual ventricular electrical activation pattern and the predetermined preferred ventricular electrical activation pattern) can be based on a single cardiac, cycle. Alternatively the determination of whether or not there is a match (between the actual ventricular electrical activation pattern and the predetermined preferred ventricular electrical activation pattern) can be based on a plurality of (e.g., N) cardiac cycles. Where based on N cardiac cycles, it can be that there needs to be a match in all of the N cardiac cycles for there to be a match. Alternatively, where based on N cardiac cycles, it can be that there needs to be a match in at least M out of the N cardiac cycles for there to be a match (where M<N). It can also be that a predominant actual sequence and an average or mean of the actual delays are determined and used in the comparisons to the predetermined preferred sequence and delays.

As indicated in steps 408 and 410, if the actual ventricular electrical activation pattern does not match the predetermined preferred ventricular electrical activation pattern, then MSLV pacing is delivered to achieve the predetermined preferred ventricular electrical activation pattern. As indicated by step 408, if the actual ventricular electrical activation pattern matches the predetermined preferred ventricular electrical activation pattern, then flow returns to step 402, and there is no need to deliver MSLV pacing at the time. This conserves battery resources, since MSLV pacing requires more power than single site LV pacing, or no LV pacing.

Assume, for example, that the predetermined preferred ventricular electrical activation pattern includes the predetermined preferred sequence LV1-LV2-RV, that the predetermined preferred LV1-LV2 delay=20 ms, and that the predetermined preferred LV2-RV delay=50 ms. If the actual ventricular electrical activation pattern does not match the predetermined preferred ventricular electrical activation pattern, then the multisite LV pacing delivered at step 410 can include pacing at the LV1 site in LV chamber, then pacing at the LV2 site in the LV chamber the predetermined preferred LV1-LV2 delay (i.e., 20 ms) after pacing the LV1 site, and then pacing in the RV chamber the predetermined preferred LV2-RV delay (i.e., 50 ms) after pacing the LV2 site. The pacing at each site should occur before the corresponding intrinsic activation at that site.

For another example, assume that the predetermined preferred ventricular electrical activation pattern includes the predetermined preferred sequence RV-LV1-LV2, that the predetermined preferred RV-LV1 delay=60 ms, and that the predetermined preferred LV1-LV2 delay=30 ms. If the actual ventricular electrical activation pattern does not match the predetermined preferred ventricular electrical activation pattern, then the multisite LV pacing delivered at step 410 can include pacing in the RV chamber, then pacing at the LV1 site in the LV chamber the predetermined preferred RV-LV1 delay (i.e., 60 ms) after pacing in the RV chamber, and then pacing at the LV2 site in the LV chamber the predetermined preferred LV1-LV2 delay (i.e., 30 ms) after pacing the LV1 site.

For a further example, assume that the predetermined preferred ventricular electrical activation pattern indicates that an activation should first occur at an LV1 region, and that activations at an LV2 region and at an RV region (or more generally, within the RV chamber) should each occur 30 ms after the activation at the LV1 activation (i.e., the predetermined preferred LV1-LV2 delay and the predetermined LV1-RV delay can both equal 30 ms). If the actual ventricular electrical activation pattern does not match the predetermined preferred ventricular electrical activation pattern, then the multisite LV pacing delivered at step 410 can include pacing at the LV1 site, and then simultaneously pacing the LV2 site in the LV chamber and at the RV region within the RV chamber at the same predetermined preferred delay (i.e., 30 ms) after pacing at the LV1 site. It can also be the predetermined preferred LV1-LV2 delay (or range of delays) differs from the predetermined LV1-RV delay (or range of delays).

Where the predetermined preferred ventricular electrical activation pattern includes the predetermined preferred sequence RV-LV1-LV2, if an intrinsic activation within the RV chamber consistently occurs before LV activation, it is also possible to allow for the intrinsic activation of the RV chamber (i.e., to not pace in the RV chamber), and then perform MSLV pacing at the LV1 and LV2 sites using the predetermined preferred RV-LV1 and LV1-LV2 delays. In other words, a predetermined preferred RV-LV1 delay can be used to time delivery of pacing at the LV1 site relative to an intrinsic RV activation.

Where the predetermined preferred ventricular electrical activation pattern includes the predetermined preferred sequence LV1-LV2-RV, if an intrinsic activation at the LV1 site consistently occurs before LV2 and RV activations, it is also possible to allow for the intrinsic activation at the LV1 site within the LV chamber (i.e., to not pace at the LV1 site), and then perform pacing at the LV2 site and within the RV chamber using the predetermined preferred LV1-LV2 and LV2-RV delays. In other words, a predetermined preferred LV1-LV2 delay can be used to time delivery of pacing at the LV2 site relative to an intrinsic activation at the LV1 site.

Depending upon the specific implementation, where the delays are defined as a range of acceptable delays, if the actual ventricular electrical activation pattern does not match the predetermined preferred ventricular electrical activation pattern, then the delays used for pacing (when the actual pattern does not match the predetermined preferred pattern) can be the lower values of the ranges, the upper values of the ranges, or an average of the ranges, but are not limited thereto. It is also possible that specific delays are programmed to be used when one or more of the actual delays is/are not within a corresponding programmed range of delays.

In accordance with certain embodiments, step 402 is performed while the patient's LV chamber is not being paced. In accordance with other embodiments, step 402 is performed during bi-ventricular (BiV) pacing that includes pacing at only a single site within the LV chamber. Either way, MSLV pacing is selectively delivered when the actual ventricular electrical activation pattern does not match the predetermined preferred ventricular electrical activation pattern.

In accordance with certain embodiments, once the MSLV pacing is initiated at step 410 it continues for a programmed duration (e.g., 10 minutes), and then returns to the normal mode, which can be no LV pacing (e.g., an intrinsic mode), or single site LV pacing (e.g., as part of BiV pacing). Thereafter, steps 402-410 can be repeated, as indicated by line 412, which can result in MSLV pacing being reinitiated at step 410. Over time, use of the embodiments described with reference to the flow diagram of FIG. 4 may achieve cardiac reverse remodeling of the ventricles that results in MSLV pacing needing to be performed at step 410 less frequently.

The timing diagram of FIG. 5 illustrates how an actual ventricular electrical activation pattern can be determined from three IEGMs, where the first IEGM is used to identify electrical activations in the RV chamber, the second IEGM is used to identify electrical activations at an LV1 site in the LV chamber, and the third IEGM is used to identify electrical activations at an LV2 site in the LV chamber. In FIG. 5, “−ACT” refers to a detected activation, e.g., a right ventricular activation is shown as RV-ACT. As can be appreciated from FIG. 5, the actual ventricular electrical activation sequence is RV-LV1-LV2. FIG. 5 also illustrates how an actual RV-LV1 delay and an actual LV1-LV2 delay can be determined.

The criterion for activation detections (based on the IEGMs) can be, e.g., a threshold-crossing against an automatic sensitivity control (ASC) level or an R-wave threshold-crossing. Alternatively, the criterion for an activation detection (based on the IEGMs) can be based on an absolute value or upward slope of an R-wave, or based on a maximum dV/dt of an R-wave. Other known or future developed criterion for detecting electrical activations are also possible, and within the scope of embodiments of the present invention.

The embodiments of the present invention described with reference to FIGS. 4 and 5 can be used to improve the cardiac mechanical function and physiology of a patient by increasing compliance with a predetermined preferred ventricular electrical activation pattern that a cardiologist or other physician determined to be beneficial. It is also believed that these embodiments can provide beneficial cardiac reverse remodeling of the ventricles.

### Arrhythmia Discrimination Based on Localized R-R Interval Stability Corresponding to a Plurality of Ventricular Regions

FIG. 6 is a high level flow diagram that is used to describe techniques to perform arrhythmia discrimination based on a plurality of R-R interval stability metrics corresponding to a plurality of different ventricular regions, according to specific embodiments of the present invention. More generally, FIG. 6 is used to describe techniques for performing interval stability (which can also be referred to as rate stability) analysis at multiple ventricular regions, in accordance with embodiments of the present invention.

Referring to FIG. 6, at step 602 a plurality of different sensing vectors are used to obtain a plurality of different IEGMs, each of which is indicative of cardiac electrical activity at a different ventricular region.

In specific embodiments, the plurality of IEGMs obtained at step 602 include an IEGM indicative of cardiac electrical activity at a first region of the patient's LV chamber, and an IEGM indicative of cardiac electrical activity at a second region of the patient's LV chamber. Such embodiments are believed to be advantageous because they enable localized stability analysis at multiple sites within a same ventricular chamber (i.e., the LV chamber), which can be compared to one another, thereby providing for enhanced arrhythmia discrimination. The time between consecutive ventricular activations at each separate ventricular region (which can also be referred to as a location or a site) can be referred to as a localized R-R interval. While the stability of R-R intervals at a single fixed location has been widely used to characterize an arrhythmia as atrial fibrillation (AF), specific embodiments of the present invention utilize multiple localized R-R intervals to characterize an arrhythmia as other types of arrhythmias in addition to AF. In other words, conventional R-R interval stability analysis has generally only been used to characterize an arrhythmia as AF or not AF. By contrast, specific embodiments of the present invention described herein can utilize multiple localized R-R intervals to characterize an arrhythmia as an SVT with rapid ventricular response, an AVRT, an AVNRT, and AF, as will be explained in additional detail below. Stated another way, certain embodiments of the present invention can perform arrhythmia discrimination, based on a plurality of localized R-R interval stability metrics, that includes more than determining whether or not an arrhythmia is AF.

The plurality of IEGMs obtained at step 602 can also include an IEGM indicative of cardiac electrical activity at a region of a patient's RV chamber.

The sensing vector used to obtain the IEGM indicative of cardiac electrical activity at a first region of a patient's LV chamber can include, e.g., the D1 electrode 126₁ configured as a cathode and the M2 electrode 126₂ (and/or the RV coil electrode 136) configures as an anode; while the sensing vector used to obtain the IEGM indicative of cardiac electrical activity at a second region of a patient's LV chamber can include, e.g., the P4 electrode 126₁ configured as a cathode and the M3 electrode 126₃ (and/or the RV coil electrode 136) configured as an anode. These are just a few examples, which are not meant to be all inclusive. The locations of cathode electrodes within the LV chamber can also be referred to as LV sites, e.g., LV1 and LV2 sites. In other words, a first region of a patient's LV chamber can correspond to an LV1 site, and a second region of the patient's LV chamber can correspond to an LV2 site.

The sensing vector used to obtain the IEGM indicative of cardiac electrical activity at a region of a patient's RV chamber preferably includes at least one electrode within the RV chamber configures as a cathode, with an electrode within and/or outside the RV chamber configured as an anode. For example, referring to FIG. 1A, one of the RV tip electrode 132 and the RV ring electrode 134 can be configured as the cathode, while the other one is configured as the anode. Alternatively, or additionally, the RV coil electrode 136 can be configured as the anode. These are just a few examples, which are not meant to be all inclusive.

At step 604, for each of the IEGMs, a corresponding localized R-R interval stability metric (indicative of the R-R interval stability at the corresponding ventricular region) is determined. A localized R-R interval stability metric can be determined for an IEGM by determining the R-R intervals for a plurality of consecutive cardiac cycles (e.g., 60 cardiac cycles, but not limited thereto) of the IEGM, and determining a measure of variation of the R-R intervals. Exemplary measures of variation, which can be determined for the R-R intervals, can include one or more of: standard deviation, normalized standard deviation, interquartile range, range, mean difference, median absolute deviation, average absolute deviation, coefficient of variation, quartile coefficient of dispersion, relative mean difference, variance, and variance-to-mean ratio of the metric, but are not limited thereto.

Continuing with the above example (where the plurality of IEGMs include an IEGM indicative of cardiac electrical activity at a region of a patient's RV chamber, an IEGM indicative of cardiac electrical activity at a first region of the patient's LV chamber, and an IEGM indicative of cardiac electrical activity at a second region of the patient's LV chamber), at step 604 one or more R-R interval stability metric is determined for each of the IEGMs. Where a plurality of R-R interval stability metrics are determined for each IEGM, such metrics can be combined, e.g., using an algorithm and/or weighting factors. For simplicity, it can be assumed that a single common localized R-R interval stability metric (e.g., standard deviation) is determined for each IEGM, but that need not be the case.

At step 606, arrhythmia discrimination is performed based on the localized R-R interval stability metrics determined at step 604, as explained in more detail below. At step 608, therapy is selecting, an alert is triggered and/or information is stored based on results of the arrhythmia discrimination, as discussed in more detail below.

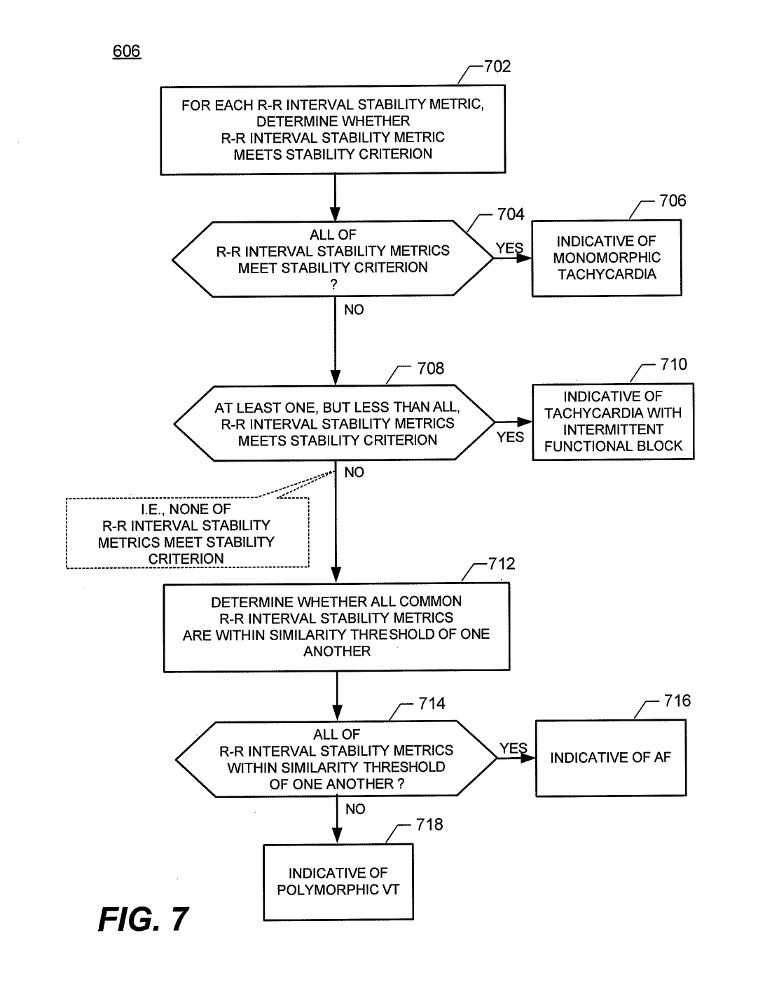

Additional details of step 606, according to specific embodiments of the present invention, are provided below with reference to the flow diagram of FIG. 7. Referring to FIG. 7, at step 702, for each of the localized R-R interval stability metrics, there is a determination of whether the localized R-R interval stability metric meets a corresponding stability criterion. In accordance with an embodiment, step 702 can include comparing each localized R-R interval stability metric (determined for an IEGM) to an appropriate R-R interval stability threshold. If the localized R-R interval stability metric (e.g., a measure of standard deviation) does not exceed the R-R interval stability threshold (e.g., a standard deviation threshold), then it can be determined that the stability criterion is met. Conversely, if the localized R-R interval stability metric (e.g., the measure of standard deviation) exceeds the R-R interval stability threshold (e.g., the standard deviation threshold), then it can be determined that the stability criterion is not met.

As can be appreciated by steps 704 and 706, if all of the localized R-R interval stability metrics meet the stability criterion, there is a determination that the localized R-R interval stability metrics are indicative of a monomorphic tachycardia (e.g., monomorphic VT). Where one or more further algorithms, e.g., a rate branch and/or morphology discrimination algorithm, is used to distinguish VT from SVT, an arrhythmia can be classified as monomorphic VT or monomorphic SVT at step 706 (e.g., depending on the results of the rate branch and/or morphology discrimination algorithm(s)). A monomorphic tachycardia results in consistent QRS complexes throughout the ventricular chambers, which are observable, in accordance with embodiments of the present invention, as stable R-R intervals occurring in all of the ventricular regions being sensed.

As can be appreciated by steps 708 and 710, if at least one of the localized R-R interval stability metrics meets the stability criterion while another one of the localized R-R interval stability metrics does not meet the interval stability criterion, there is a determination that the localized R-R interval stability metrics are indicative of a tachycardia with intermittent functional block. Where one or more further algorithms, e.g., a rate branch and/or morphology discrimination algorithm(s), is used to distinguish VT from SVT, an arrhythmia can be classified as VT with intermittent functional block or SVT with intermittent functional block at step 710 (e.g., depending on the results of the rate branch and/or morphology discrimination algorithm(s)).

If it is determined at step 708 that none of the localized R-R interval stability metrics meets the stability criterion (i.e., if the answer to step 708 is no), then there is a determination at step 712 of whether or not all of the localized R-R interval stability metrics (that do not meet the stability criterion) are within a similarity threshold of one another. This can include, e.g., determining whether the localized R-R interval stability metrics (e.g., standard deviation) determined from the plurality of sense IEGMs are within a specified tolerance (e.g., 10%) of one another.

As can be appreciated by steps 714 and 716, if all of the localized R-R interval stability metrics do not meet the stability criterion, and all of the localized R-R interval stability metrics are within a similarity threshold of one another, then there is a determination that the localized R-R interval stability metrics are indicative of atrial fibrillation (AF) with fast irregular atrio-ventricular (AV) conduction.

As can be appreciated by steps 714 and 718, if all of the localized R-R interval stability metrics do not meet the stability criterion, and all of localized R-R interval stability metrics are not within the similarity threshold of one another, then there is a determination that the localized R-R interval stability metrics are indicative of polymorphic VT. A polymorphic VT is often caused by abnormalities of ventricular muscle repolarization. A polymorphic VT results in inconsistent QRS complexes throughout the ventricular chambers, which are observable, in accordance with embodiments of the present invention, as unstable and dissimilar localized R-R intervals occurring at the various ventricular regions being sensed.

In specific embodiments, steps 702-718 are only performed if a rate metric indicative of cardiac rate meets a tachycardia threshold, but does not meet a ventricular fibrillation (VF) rate threshold. For example, the rate metric can be in average beats per minute (bpm), the tachycardia threshold can be a bpm tachycardia threshold (e.g., 120 bpm) that if exceeded is indicative of a tachycardia, and the VF rate threshold can be a bpm tachycardia threshold (e.g., 240 bpm) that if exceeded is indicative of VF. For another example, the rate metric can be an average R-R interval, the tachycardia threshold can be a R-R interval tachycardia threshold (e.g., 0.5 seconds) that if the average R-R interval is below is indicative of a tachycardia, and the VF rate threshold can be an R-R interval threshold (e.g., 0.25 seconds) that if the average R-R interval is below is indicative of VF. Other VF detection techniques are also possible. If VF is diagnosed, then defibrillation therapy can be delivered, in an attempt to convert the VF to a normal sinus rhythm. Defibrillation therapy can include, e.g., delivery of one or more defibrillation shocks, but is not limited thereto.

The above described embodiments involving interval stability analysis for a plurality of different ventricular regions can be used as the sole technique for arrhythmia discrimination, or more likely, as one of a few or many arrhythmia discrimination techniques, some of which are discussed herein, including morphology arrhythmia discrimination and sudden onset arrhythmia discrimination. In other words, the above described interval stability analysis can be used to independently discriminate various types of arrhythmias, or can be used together with other techniques for performing arrhythmia discrimination. For example, the above described interval stability analysis embodiments can be used to supplement (e.g., to increase the confidence level of) arrhythmia discrimination performed using some other technique(s), including but not limited to sudden onset and morphology discrimination techniques. Alternatively, some other technique(s) can be used to supplement the arrhythmia discrimination performed using an above described interval stability analysis embodiment. It is also possible that the above described interval stability analysis be used in one or more branch of rate branch algorithm. Some exemplary details of interval sudden onset and morphology discrimination techniques are provided below, for completeness. Where multiple arrhythmia techniques are used, they can be use serially and/or in parallel, and various results can be combined, e.g., using algorithms, rules and/or weighting factors.

Sudden onset arrhythmia discrimination techniques can be used to assist in distinguishing between VT and a sinus tachycardia type SVT that is due to exercise (e.g., walking up a flight of stairs). Typically, a sinus tachycardia has a gradual rate of onset, while VT has a more abrupt onset. Such onset can be measured, e.g., by determining a difference between the average RR interval for N beats prior to a first beat that exceeds the tachycardia detection rate, and the average R-R interval for N beats following the first beat that exceeds the tachycardia detection rate (e.g., N can be 1 or more). Accordingly, the value of a sudden onset discriminator parameter can be specified in milliseconds. Where the sudden onset discriminator value is exceeded, the implantable cardiac device interprets that as an indicator of VT. Where a sudden onset discriminator value is not exceeded, the implantable cardiac device interprets that as an indicator of SVT.

Morphology arrhythmia discrimination techniques can be also be used to assist in discrimination between VT and SVT, because SVTs originate in the atria and follow the normal conduction pathway to the ventricles (typically via the AV node), causing the morphology (shape) of the resulting QRS complexes to look similar to the morphology of a QRS complex of a normal sinus rhythm. In contrast, VT arises from outside normal conduction system, causing the morphology of the resulting QRS complex to be less similar to that of a normal sinus rhythm. To perform such morphology comparisons, a template QRS complex is typically obtained and stored when a patient is known to have a normal sinus rhythm. Thereafter, the template QRS complex can be compared to present QRS complexes in real or near real time, to determine a level of similarity. A morphology discriminator parameter can specify, e.g., the level of similarity below which a rhythm is classified as indicative of VT, and above which the rhythm is classified as indicative of SVT. For a more specific example, a morphology algorithm can measure attributes such as the number of peaks, amplitude of peaks, polarity, and area under curves of a QRS complex, and compares such complexes to the template QRS complex to generate a percent match between 0 and 100%. For this example, a morphology discriminator parameter can specify the percentage match, above which the implantable cardiac device interprets as indicative of SVT, and below which the device interprets as indicative of VT.

Referring again to FIG. 6, at step 606 an arrhythmia may be classified, e.g., as a monomorphic VT, a monomorphic SVT, a polymorphic VT, a VT with intermittent functional block, an SVT with intermittent functional block, an AF with fast irregular AF conductions, but is not limited thereto. Based on the results of step 606, therapy is selected, an alert is triggered and/or information is stored at step 608. For example, at step 608 information indicative of the classification of an arrhythmia can be stored for later analysis by medical personnel. Additionally, or alternatively, at step 608 one or more of the plurality of IEGMs corresponding to the period of time which the arrhythmia is detected can also be stored for later analysis. Classification of specific tachycardias can be used to trigger an alert that is detectable by the patient and/or is communicated to medical personnel at step 608. Additionally, at step 608 information can be saved about therapies delivered in response to detection and/or diagnosis of an arrhythmia, and the electrical and physiologic responses to such therapies.

At step 608, a therapy can be selected based on the classification of an arrhythmia. In certain embodiments, regardless of the type of VT diagnosis, the same type of therapy, e.g., ventricular anti-tachycardia pacing (V-ATP), can be performed. In other embodiments, a first type of therapy (e.g., V-ATP) can be performed if an arrhythmia is classified as a monomorphic VT, a second type of therapy (e.g., cardioversion shock therapy) can be performed if an arrhythmia is classified as a polymorphic VT, and a third type of therapy (or the first type of therapy, e.g., V-ATP) can be performed if an arrhythmia is classified as a VT with intermittent functional block. It is also possible that one or more specific type(s) of VT is/are not treated. Other variations are also possible while being within the spirit and scope of the present invention.

In certain embodiments, regardless of the type of SVT diagnosis, the same type of therapy can be performed. In other embodiments, a first type of therapy can be performed if an arrhythmia is classified as a monomorphic SVT, a second type of therapy can be performed if an arrhythmia is classified as a polymorphic SVT, a third type of therapy can be performed if an arrhythmia is classified as an SVT with intermittent functional block, and a fourth type of therapy can be performed if an arrhythmia is classified as AF with fast irregular AV conduction. It is also possible the two or more types of SVT are treated with the same type of therapy. It is also possible that one or more specific type(s) of SVT is/are not treated. Other variations are also possible while being within the spirit and scope of the present invention. Exemplary types of therapy that can be used to treat an SVT include, but are not limited to, atrial anti-tachycardia pacing (A-ATP), atrial defibrillation, and delivering one or more premature timed extrastimulus in an atrium and/or ventricle.

### Arrhythmia Discrimination Based on Atrial and Ventricular Activation Times

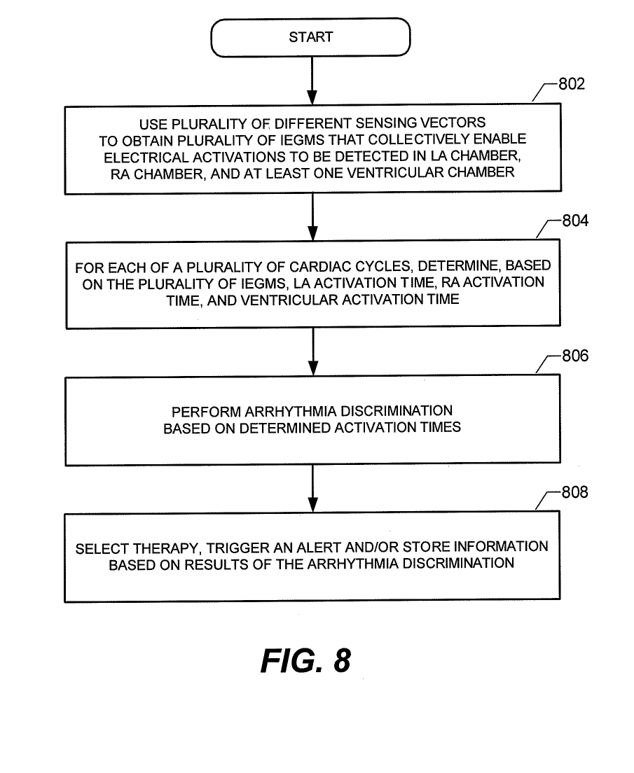

FIG. 8 is a high level flow diagram that is used to describe techniques to perform arrhythmia discrimination based on atrial and ventricular activation times, according to embodiments of the present invention.

Referring to FIG. 8, at step 802, a plurality of different sensing vectors are used to obtain a plurality of IEGMs that collectively enable electrical activations to be detected in the left atrial (LA) chamber, the right atrial (RA) chamber, and at least one ventricular chamber of a patient's heart.

Referring back to FIG. 1A, assuming that the P4 electrode 126₄ is implanted at or in the immediate proximity of the atrioventricular (AV) groove (also known as the coronary sulcus), a sensing vector that includes the P4 electrode 126₄ connected as a cathode can be used to sense electrical activations in the LA chamber as well as in the LV chamber. Additionally, a sensing vector including, e.g., the D1 electrode 126₁ (or another one of the LV electrodes) connected as a cathode can be used to specifically detect electrical activations in the LV chamber. Additionally, or alternatively, a sensing vector that includes the RV tip electrode 132 or the RV ring electrode 134 connected as a cathode can be used to detect electrical activations in the RV chamber. For each of the above sensing vectors, the anode electrode can be, e.g., the RV coil electrode 136, the case electrode 140, or the SVC coil electrode 138, but is not limited thereto, but is not limited thereto.

A sensing vector that includes RA tip electrode 122 or the RA ring electrode 123 electrode connected as a cathode can be used to detect electrical activations in the RA chamber. For such a sensing vector, RA ring electrode 123, the SVC coil electrode 138, the case electrode 140 or the RV coil electrode 136 can be connected as the anode. Other alternatives are also possible.

Returning to the flow diagram of FIG. 8, at step 804, for each of a plurality of cardiac cycles, an LA activation time, an RA activation time, and a ventricular activation time are determined based on the plurality of obtained IEGMs. For example, an LA activation time, an RA activation time, and an LV and/or RV activation time can be determined for twenty (or some other number of) consecutive cardiac cycles.

At step 806, arrhythmia discrimination is performed based on the activation times determined at step 804, as explained in more detail below. At step 808, therapy is selected, an alert is triggered and/or information is stored based on results of the arrhythmia discrimination, as discussed in more detail below.

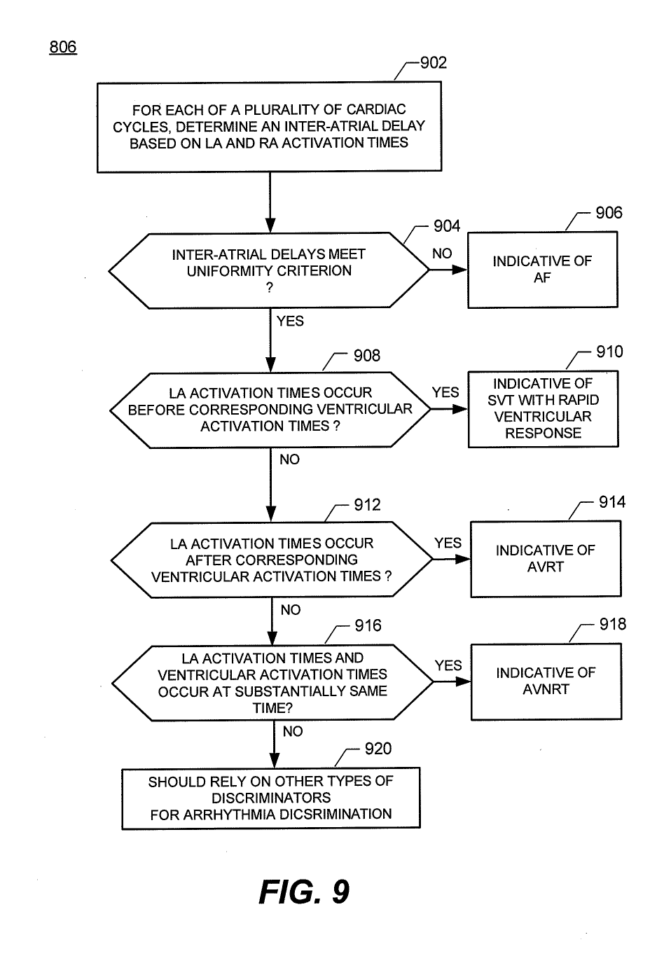

Additionally details of step 806, according to specific embodiments of the present invention, shall now be described with reference to the flow diagram of FIG. 9. Referring to FIG. 9, at step 902 there is a determination, for each of the plurality of cardiac cycles, of an inter-atrial delay based on the LA and RA activation times. At step 904 there is a determination whether the inter-atrial delays meet a uniformity criterion.

In specific embodiments, step 904 can include determining beat-to-beat differences in the inter-atrial delays, and determining, based on the beat-to-beat differences in inter-atrial delays, whether the inter-atrial delays meet the uniformity criterion. For an example, assume that inter-atrial delays are determined for twenty consecutive cardiac cycles, resulting in the twenty inter-atrial delays LA-RA delay₁, LA-RA delay₂ . . . LA-RA₂₀. The beat-to-beat differences in inter-atrial delays can be determined by determining the difference (also referred to as “the delta”) between the LA-RA delay₁ and the LA-RA delay₂; determining the difference between the LA-RA delay₂ and the LA-RA delay₃; determining the difference between the LA-RA delay₃ and the LA-RA delay₄; . . . and determining the difference between the LA-RA delay₂₀ and the LA-RA delay₁₉.

In an embodiment, if all of the beat-to-beat differences in inter-atrial delays are less than a beat-to-beat difference threshold, then it is determined that the inter-atrial delays meet the uniformity criterion; if one or more of the beat-to-beat differences in inter-atrial delays is greater than the beat-to-beat difference threshold, then it is determined that the inter-atrial delays do not meet the uniformity criterion. In an alternative embodiment, if at least N out of M (e.g., 18 out of 20) or X % (e.g., 90%) of the beat-to-beat differences in inter-atrial delays are less than a beat-to-beat difference threshold, then it is determined that the inter-atrial delays meet the uniformity criterion; otherwise it is determined that the inter-atrial delays do not meet the uniformity criterion. Other variations are also possible.

In other embodiments, step 904 can include determining a measure of variation for the beat-to-beat differences in inter-atrial delays. If the measure of variation does not exceed a beat-to-beat difference variation threshold, then it is determined that the inter-atrial delays meet the uniformity criterion. If the measure of variation exceeds the beat-to-beat difference variation threshold, then it is determined that the inter-atrial delays do not meet the uniformity criterion. Exemplary measures of variation, which can be determined for beat-to-beat differences in inter-atrial delays, can include one or more of: standard deviation, normalized standard deviation, interquartile range, range, mean difference, median absolute deviation, average absolute deviation, coefficient of variation, quartile coefficient of dispersion, relative mean difference, variance, and variance-to-mean ratio of the metric, but are not limited thereto.

As can be appreciated from steps 904 and 906, the inter-atrial delays are indicative of atrial fibrillation (AF) if the inter-atrial delays do not meet the uniformity criterion.

If it is determined at step 904 that the inter-atrial delays meet the uniformity criterion, then at step 908 there is a determination of whether each of the LA activation times occur before a corresponding ventricular activation time. In a specific embodiment, at step 908 there is a determination of whether each of the LA activation times occur at least a predetermined delay (e.g., 20 ms) before a corresponding ventricular activation time. Such a predetermined delay can be programmed to be equal to or less than a PR delay observed by a physician from a surface electrocardiogram (ECG), but is not limited thereto.

As can be appreciated from steps 908 and 910, if a specified amount (e.g., all, N out of M, or X %) of the LA activation times occur before (or at least the predetermined delay before) the corresponding ventricular activation time, then it is determined that the LA and ventricular activation times are indicative of a supraventricular tachycardia (SVT) with a rapid ventricular response, whether from normal conduction over the AV node or from fast A-V conduction over an accessory pathway. Atrial flutter (AFL) with a rapid ventricular response is an example of an SVT with a rapid ventricular response that often conducts forward over the AV node. The specified amount (e.g., all, N out of M, or X %) mentioned above can be programmed into a device by medical person, or can be hardcoded into the device.

As can be appreciated from steps 912 and 914, if a specified amount (e.g., all, N out of M, or X %) of the LA activation times occur after (or at least a predetermined delay after) corresponding ventricular activation times, then it is determined that the LA and ventricular activation times are indicative of an atrioventricular reentrant tachycardia (AVRT). Such an AVRT may be mediated by V-A conduction over an accessory pathway.

As can be appreciated from steps 916 and 918, if the LA activation times occur at substantially the same time as corresponding ventricular activation times (e.g., within a specified tolerance, such as within 3 ms), then it is determined that the LA and ventricular activation times are indicative of an atrioventricular nodal reentry tachycardia (AVNRT), which is a common type of reentrant SVT. In AVNRT, a reentry circuit is contained entirely within the AV node.

If the answers the inquiries at decision blocks 908, 912 and 916 are all no, then it may be that there are multiple tachycardias occurring simultaneously, e.g., one of atrial origin and one of ventricular origin. It may also be that the arrhythmia is very complex and/or has characteristics of several different tachycardias. In this case, other algorithms may be needed to characterize the arrhythmia(s), as indicated by block 920.

In specific embodiments, steps 804 and 806 (including steps 902-914) are only performed if a rate metric indicative of cardiac rate meets a tachycardia threshold, but does not meet a ventricular fibrillation (VF) rate threshold. For example, the rate metric can be in average beats per minute (bpm), the tachycardia threshold can be a bpm tachycardia threshold (e.g., 120 bpm) that if exceeded is indicative of a tachycardia, and the VF rate threshold can be a bpm tachycardia threshold (e.g., 240 bpm) that if exceeded is indicative of VF. For another example, the rate metric can be an average R-R interval, the tachycardia threshold can be an R-R interval tachycardia threshold (e.g., 0.5 seconds) that if the average R-R interval is below is indicative of a tachycardia, and the VF rate threshold can be an R-R interval threshold (e.g., 0.25 seconds) that if the average R-R interval is below is indicative of VF. Other VF detection techniques are also possible. If VF is diagnosed, then defibrillation therapy can be delivered, in an attempt to convert the VF to a normal sinus rhythm. Defibrillation therapy can include, e.g., delivery of defibrillation shocks, but is not limited thereto. In certain embodiments, steps 804 and 806 (including steps 902-914) are only performed once an arrhythmia has been generally classified as an SVT (and thus, not as VT or VF).

An above described technique for performing arrhythmia discrimination based on atrial and ventricular activation times can be used as the sole technique for arrhythmia discrimination, or more likely, as one of a few or many arrhythmia discrimination techniques, some of which were already discussed above (in the discussion of FIGS. 6 and 7), including morphology arrhythmia discrimination and sudden onset arrhythmia discrimination. In other words, the above described arrhythmia discrimination based on atrial and ventricular activation times can be used to independently discriminate various types of arrhythmias, or can be used together with other techniques for performing arrhythmia discrimination. For example, the above described embodiments that perform arrhythmia discrimination based on atrial and ventricular activation times can be used to supplement (e.g., to increase the confidence level of) arrhythmia discrimination performed using some other technique(s), including but not limited to sudden onset and morphology discrimination techniques. Alternatively, some other technique(s) can be used to supplement the above described embodiments that perform arrhythmia discrimination based on atrial and ventricular activation times. It is also possible that one of the above described embodiments that perform arrhythmia discrimination based on atrial and ventricular activation times be used in one or more branch of rate branch algorithm. Where multiple arrhythmia techniques are used, they can be use serially and/or in parallel, and various results can be combined, e.g., using algorithms, rules and/or weighting factors. Some exemplary details of interval sudden onset and morphology discrimination techniques were provided above (in the discussion of the embodiments of FIGS. 6 and 7), for completeness.

Referring again to FIG. 8, at step 806 an arrhythmia may be classified, e.g., as AF, SVT with rapid ventricular response, AVRT, or AVNRT, but is not limited thereto. Based on the results of step 806, therapy can be selected, an alert can be triggered and/or information can be stored at step 808. For example, at step 808 information indicative of the classification of an arrhythmia can be stored for later analysis by medical personnel. Additionally, or alternatively, at step 808 one or more of the plurality of IEGMs corresponding to the period of time which the arrhythmia is detected can also be stored for later analysis. Classification of specific tachycardias can be used to trigger an alert that is detectable by the patient and/or is communicated to medical personnel at step 808. Additionally, at step 808 information can be saved about therapies delivered in response to detection and/or diagnosis of an arrhythmia, and the electrical and physiologic responses to such therapies.

At step 808, a therapy can be selected based on the classification of an arrhythmia. In certain embodiments, regardless of the type of SVT diagnosis, the same type of therapy, e.g., atrial anti-tachycardia pacing (A-ATP), can be performed. In other embodiments, a first type of therapy can be performed if an arrhythmia is classified as AF, a second type of therapy can be performed if an arrhythmia is classified as SVT with rapid ventricular response, and a third type of therapy can be performed if an arrhythmia is classified as AVNRT. It is also possible that one or more specific type(s) of SVT (e.g., AF) is/are not treated. Other variations are also possible while being within the spirit and scope of the present invention.

Embodiments of the present invention have been described above with the aid of functional building blocks illustrating the performance of specified functions and relationships thereof. The boundaries of these functional building blocks have often been arbitrarily defined herein for the convenience of the description. Alternate boundaries can be defined so long as the specified functions and relationships thereof are appropriately performed. Any such alternate boundaries are thus within the scope and spirit of the claimed invention. For example, it would be possible to combine or separate some of the steps shown in FIGS. 2, 4, 6, 7, 8 and 9. For another example, it is possible to change the boundaries of some of the blocks shown in FIG. 1B.

The previous description of the preferred embodiments is provided to enable any person skilled in the art to make or use the embodiments of the present invention. While the invention has been particularly shown and described with reference to preferred embodiments thereof, it will be understood by those skilled in the art that various changes in form and details may be made therein without departing from the spirit and scope of the invention.

What is claimed is:

1. For use with an implantable system including a first lead having at least one electrode implantable within a right ventricular (RV) chamber, and a second lead having at least two electrodes implantable within a left ventricular (LV) chamber, a method for increasing compliance with a predetermined preferred ventricular electrical activation pattern, the method comprising:
(a) using a plurality of different sensing vectors to obtain a plurality of intracardiac electrograms (IEGMs) that collectively enable electrical activations to be detected in at least the RV chamber and at at least two separate regions of the LV chamber;
(b) determining an actual ventricular electrical activation pattern based on the plurality of IEGMs;
(c) determining whether the actual ventricular electrical activation pattern matches the predetermined preferred ventricular electrical activation pattern; and
(d) delivering pacing that includes multisite LV (MSLV) pacing to achieve the predetermined preferred ventricular electrical activation pattern, if the actual ventricular electrical activation pattern does not match the predetermined preferred ventricular electrical activation pattern.

2. The method of claim 1, wherein the predetermined preferred ventricular electrical activation pattern includes:
a predetermined preferred ventricular electrical activation sequence; and
at least two predetermined preferred ventricular electrical activation delays that include at least one predetermined preferred intra-ventricular delay.

3. The method of claim 2, wherein the at least two predetermined preferred ventricular electrical activation delays include a predetermined preferred inter-ventricular delay.

4. The method of claim 3, wherein:
the predetermined preferred ventricular electrical activation sequence comprises an RV-LV1-LV2 sequence; and
the predetermined preferred ventricular electrical activation delays comprise a predetermined preferred RV-LV1 delay and a predetermined preferred LV1-LV2 delay, or a range of predetermined preferred RV-LV1 delays and a range of predetermined preferred LV1-LV2 delays.

5. The method of claim 4, wherein step (d) comprises the following, if the actual ventricular electrical activation pattern does not match the predetermined preferred ventricular electrical activation pattern:
(d.1) pacing in the RV chamber;
(d.2) pacing at the LV1 site in the LV chamber the predetermined preferred RV-LV1 delay after pacing in the RV chamber at step (d.1); and
(d.3) pacing at the LV2 site in the LV chamber the predetermined preferred LV1-LV2 delay after pacing at the LV1 site at step (d.2).

6. The method of claim 3, wherein:
the predetermined preferred ventricular electrical activation sequence comprises an LV1-LV2-RV sequence; and
the predetermined preferred ventricular electrical activation delays comprise a predetermined preferred LV1-L2 delay and a predetermined preferred LV2-RV delay, or a range of predetermined preferred LV1-LV2 delays and a range of predetermined preferred LV2-RV delays.

7. The method of claim 6, wherein step (d) comprises the following, if the actual ventricular electrical activation pattern does not comply with the predetermined preferred ventricular electrical activation pattern:
(d.1) pacing at the LV1 site in LV chamber;
(d.2) pacing at the LV2 site in the LV chamber the predetermined preferred LV1-LV2 delay after pacing at the LV1 site at step (d.1); and
(d.3) pacing in the RV chamber the predetermined preferred LV2-RV delay after pacing at the LV2 site at step (d.2).

8. The method of claim 1, wherein step (a) is performed while the LV chamber is not being paced, or during bi-ventricular (BiV) pacing that includes pacing at only a single site within the LV chamber.

9. The method of claim 1, wherein:
the predetermined preferred ventricular electrical activation pattern includes a predetermined preferred ventricular electrical activation sequence and
at least two predetermined preferred ventricular electrical activation delays or ranges of delays;
step (b) includes determining an actual ventricular electrical activation sequence and at least two actual ventricular electrical activation delays corresponding to the actual ventricular electrical activation pattern; and
step (c) includes
determining that the actual ventricular electrical activation pattern matches the predetermined preferred ventricular electrical activation pattern if
the actual ventricular electrical activation sequence is the same as the predetermined preferred ventricular electrical activation sequence, and
the actual ventricular electrical activation delays are within a predetermined tolerance of, or within the ranges of, the corresponding predetermined preferred ventricular electrical activation delays,
otherwise determining that the actual electrical activation pattern does not match the predetermined preferred ventricular electrical activation pattern.

10. The method of claim 1, wherein step (a) comprises:
(a.1) using a first sensing vector having a cathode within the RV chamber to obtain a first IEGM that enables electrical activations to be detected in the RV chamber;
(a.2) using a second sensing vector having a cathode at a first LV (LV1) site within the LV chamber to obtain a second IEGM that enables electrical activations to be detected at a first LV (LV1) site in the LV chamber; and
(a.3) using a third sensing vector having a cathode at a second LV (LV2) site within the LV chamber to obtain a third IEGM that enables electrical activations to be detected at a second LV (LV2) site in the LV chamber.

11. An implantable system, comprising:
a first lead having at least one electrode implantable in a right ventricular (RV) chamber;
a second lead having at least two electrodes implantable in a left ventricular (LV) chamber;
one or more sensing circuits configured to sense cardiac electrical activity;
one or more pulse generator configured to produce pacing pulses; and
a sensing vector controller configured to selectively connect subsets of the electrodes to a said sensing circuit to thereby provide a plurality of sensing vectors that enable a plurality of different intracardiac electrograms (IEGMs) to be obtained that collectively enable electrical activations to be detected in at least the RV chamber and at at least two separate regions of the LV chamber;
an electrical activation pattern monitor configured to
determine an actual ventricular electrical activation pattern based on the plurality of IEGMs; and
determine whether the actual ventricular electrical activation pattern matches a predetermined preferred ventricular electrical activation pattern; and
a pacing controller configured to selectively connect subsets of the electrodes to a said pulse generator to deliver pacing that includes multisite LV (MSLV) pacing to achieve the predetermined preferred ventricular electrical activation pattern, if the electrical activation pattern monitor determines that the actual ventricular electrical activation pattern does not match the predetermined preferred ventricular electrical activation pattern.

12. The implantable system of claim 11, wherein the predetermined preferred ventricular electrical activation pattern includes:
a predetermined preferred ventricular electrical activation sequence; and
at least two predetermined preferred ventricular electrical activation delays that include at least one predetermined preferred intra-ventricular delay.

13. The implantable system of claim 12, wherein the at least two predetermined preferred ventricular electrical activation delays include a predetermined preferred inter-ventricular delay.

14. The implantable system of claim 13, wherein:
the predetermined preferred ventricular electrical activation sequence comprises an RV-LV1-LV2 sequence; and
the predetermined preferred ventricular electrical activation delays comprise a predetermined preferred RV-LV1 delay and a predetermined preferred LV1-LV2 delay, or a range of predetermined preferred RV-LV1 delays and a range of predetermined preferred LV1-LV2 delays.

15. The implantable system of claim 14, wherein, if the actual ventricular electrical activation pattern does not match the predetermined preferred ventricular activation pattern, the pacing controller is configured to deliver pacing to achieve the predetermined preferred ventricular electrical activation pattern by:
pacing in the RV chamber;
pacing at the LV1 site in the LV chamber the predetermined preferred RV-LV1 delay after pacing the RV chamber; and
pacing at the LV2 site in the LV chamber the predetermined preferred LV1-LV2 delay after pacing the LV1 site.

16. The implantable system of claim 13, wherein:
the predetermined preferred ventricular electrical activation sequence comprises an LV1-LV2-RV sequence; and
the predetermined preferred ventricular electrical activation delays comprise a predetermined preferred LV1-L2 delay and a predetermined preferred LV2-RV delay, or a range of predetermined preferred LV1-LV2 delays and a predetermined preferred LV2-RV delays.

17. The implantable system of claim 16, wherein, if the actual ventricular electrical activation pattern does not match the predetermined preferred ventricular activation pattern, the pacing controller is configured to deliver pacing to achieve the predetermined preferred ventricular electrical activation pattern by:
pacing at the LV1 site in LV chamber;
pacing at the LV2 site in the LV chamber the predetermined preferred LV1-LV2 delay after step pacing at the LV1 site; and
pacing in the RV chamber the predetermined preferred LV2-RV delay after pacing at the LV2 site.

18. The implantable system of claim 11, wherein the IEGMs, which are used to determine the actual ventricular electrical activation pattern, are obtained while the LV chamber is not being paced, or during bi-ventricular (BiV) pacing that includes pacing at only a single site within the LV chamber.

19. The implantable system of claim 11, wherein the predetermined preferred ventricular electrical activation pattern includes a predetermined preferred ventricular electrical activation sequence and at least two predetermined preferred ventricular electrical activation delays or ranges of delays; and wherein the electrical activation pattern monitor is configured to:
determine an actual ventricular electrical activation sequence and at least two actual ventricular electrical activation delays corresponding to the actual ventricular electrical activation pattern; and
determining that the actual ventricular electrical activation pattern matches the predetermined preferred ventricular electrical activation pattern if
the actual ventricular electrical activation sequence is the same as the predetermined preferred ventricular electrical activation sequence, and
the actual ventricular electrical activation delays are within a predetermined tolerance of, or within the range of, the corresponding predetermined preferred ventricular electrical activation delays,
otherwise the electrical activation pattern monitor will determine that the actual electrical activation pattern does not match the predetermined preferred ventricular electrical activation pattern.

20. The implantable system of claim 11, the sensing vector controller selectively connects subsets of the electrodes to a said sensing circuit to thereby provide:
a first sensing vector having a cathode within the RV chamber and used to obtain a first IEGM that enables electrical activations to be detected in the RV chamber;
a second sensing vector having a cathode at a first site within the LV chamber and used to obtain a second IEGM that enables electrical activations to be detected at a first LV site (LV1) in the LV chamber; and
a third sensing vector having a cathode at a second site within the LV chamber and used to obtain a third IEGM that enables electrical activations to be detected at a second LV site (LV2) in the LV chamber.
